(19) United States
(12) Patent Application Publication
(10) Pub. No.: US 2026/0171197 A1
(43) Pub. Date: Jun. 18, 2026

(54) ENVIRONMENTAL ATTRIBUTES FOR CARE COMPOSITION INGREDIENTS

(71) Applicant: BASF SE, Ludwigshafen Am Rhein (DE)

(72) Inventors: Bastian GRUMBRECHT, Ludwigshafen (DE); Steffen KLOSTERHALFEN, Ludwigshafen (DE); Christian KRUEGER, Ludwigshafen (DE); Ana ALBA PEREZ, Ludwigshafen (DE); Martin BINDER, Ludwigshafen (DE); Alessandro PISTILLO, Ludwigshafen (DE); Christopher Alec ANDERLOHR, Ludwigshafen (DE); Kurt WENZL, Ludwigshafen (DE)

(21) Appl. No.: 18/711,805

(22) PCT Filed: Dec. 19, 2022
(86) PCT No.: PCT/EP2022/086810

§ 371 Date: May 20, 2024

Related U.S. Application Data
(60) Provisional application No. 63/416,091, filed on Oct. 14, 2022.

(30) Foreign Application Priority Data
Dec. 21, 2021 (EP) 21216268.9
Dec. 21, 2021 (EP) 21216269.7
Dec. 21, 2021 (EP) 21216270.5
Dec. 21, 2021 (EP) 21216271.3
Dec. 21, 2021 (EP) 21216286.1
Dec. 21, 2021 (EP) 21216292.9
Dec. 21, 2021 (EP) 21216326.5
Dec. 21, 2021 (EP) 21216327.3
Dec. 21, 2021 (EP) 21216333.1
Apr. 4, 2022 (EP) 22166573.0
Apr. 12, 2022 (EP) 22167945.9
May 10, 2022 (EP) 22172609.4
May 10, 2022 (EP) 22172611.0
May 10, 2022 (EP) 22172615.1
May 10, 2022 (EP) 22172617.7
May 10, 2022 (EP) 22172619.3
Sep. 9, 2022 (EP) 22194793.0
Sep. 9, 2022 (EP) 22194800.3
Sep. 9, 2022 (EP) 22194808.6
Sep. 9, 2022 (EP) 22194815.1
Sep. 9, 2022 (EP) 22194818.5
Oct. 14, 2022 (EP) 22201672.7
Oct. 18, 2022 (EP) 22202183.4
Dec. 5, 2022 (EP) 22211421.7

(51) Int. Cl. G16C 20/50

(52) U.S. Cl. CPC G16C 20/50

(57) ABSTRACT

Disclosed herein are systems for producing a care composition ingredient associated with a digital asset, methods for producing a care composition ingredient associated with a digital asset, apparatuses for generating a digital asset, computer-implemented methods for generating a chemical passport, computer program elements for generating a digital asset, uses of a care composition ingredient associated with a digital asset, uses of a digital asset, products produced from the care composition ingredient and associated with a digital asset, a digital asset including one or more decentral identifier(s) and data related to the environmental impact data, apparatuses for producing a product associated with the digital asset and methods for producing a product associated with the digital asset.

### TECHNICAL FIELD

The present disclosure relates to systems for producing a care composition ingredient associated with a care composition ingredient passport or digital asset, methods for producing a care composition ingredient associated with a care composition ingredient passport or digital asset, apparatuses for generating a passport or digital asset associated with a [care composition ingredient, computer-implemented methods for generating a care composition ingredient passport or digital asset, computer program elements for generating a care composition ingredient passport or digital asset, uses of a care composition ingredient associated with a care composition ingredient passport or digital asset, uses of a care composition ingredient passport or digital asset, products produced from the care composition ingredient and associated with a care composition ingredient passport or digital asset, a care composition ingredient, a care composition ingredient passport or digital asset including one or more decentral identifier(s) and data related to environmental impact data, apparatuses for producing a product associated with the care composition ingredient passport or digital asset and methods for producing a product associated with the care composition ingredient passport or digital asset.

### TECHNICAL BACKGROUND

In supply chains the environmental impact of each supply chain participants is of great interest. Specifically in the field of chemistry, care composition ingredients are employed for a wide range of applications and are supplied to diverse value chains. In such complex systems transparency between value chain participants is hard to achieve.

### SUMMARY OF THE INVENTION

In one aspect disclosed is a system for producing a care composition ingredient associated with a care composition ingredient passport or a digital asset, the system comprising:
a chemical production network configured to produce the care composition ingredient wherein the care composition ingredient is produced from one or more input material(s) through one or more chemical process(es) of the chemical production network, wherein the one or more input material(s) and/or the one or more chemical process(es) are associated with environmental attribute(s);
a production operating apparatus configured to generate the care composition ingredient passport or digital asset by
   providing a decentral identifier associated with the produced care composition ingredient and the one or more environmental attribute(s) of the one or more input material(s) and/or the one or more chemical process(es);
   linking the decentral identifier and the environmental attribute(s);
optionally the chemical production network or system configured to provide the produced care composition ingredient in association with the care composition ingredient passport or digital asset.

In another aspect disclosed is a system for producing a care composition ingredient associated with a care composition ingredient passport or digital asset, the system comprising:
a chemical production network configured to produce the care composition ingredient from one or more input material(s) through chemical process(es), wherein the one or more input material(s) and/or the chemical process(es) are associated with environmental attribute(s);
a production operating apparatus configured to generate the care composition ingredient passport or digital asset by providing and/or linking a decentral identifier associated with the care composition ingredient and one or more environmental attribute(s) associated with the one or more input material(s) and/or the chemical process(es).

In another aspect disclosed is a method for producing a care composition ingredient associated with a care composition ingredient passport or digital asset, wherein the method comprises:
producing the care composition ingredient from one or more input material(s) through one or more chemical process(es) of a chemical production network, wherein the one or more input material(s) and/or the one or more chemical process(es) are associated with environmental attribute(s);
generating the care composition ingredient passport or digital asset by
   providing a decentral identifier associated with the produced care composition ingredient and one or more environmental attribute(s) associated with the one or more input material(s) and/or the one or more chemical process(es);
   linking the decentral identifier and the one or more environmental attribute(s);
providing the produced care composition ingredient in association with the care composition ingredient passport or digital asset.

In another aspect disclosed is a method for producing a care composition ingredient associated with a care composition ingredient passport or a digital asset, wherein the method comprises:
producing the care composition ingredient from one or more input material(s) through one or more chemical process(es) of a chemical production network, wherein the one or more input material(s) and/or the one or more chemical process(es) are associated with environmental attribute(s),
generating the care composition ingredient passport or digital asset by providing and/or linking a decentral identifier associated with the care composition ingredient and one or more environmental attribute(s) of the one or more input material(s) and/or the chemical process(es).

In another aspect disclosed is an apparatus for generating a passport or digital asset associated with a care composition ingredient, wherein the care composition ingredient is produced from one or more input material(s) through one or more chemical process(es) of a chemical production network, wherein the one or more input material(s) and/or the one or more chemical process(es) are associated with environmental attribute(s), the apparatus comprising:
a decentral identity provider configured to provide a decentral identifier associated with the produced care composition ingredient and one or more environmental attribute(s) of the one or more input material(s) and/or the one or more chemical process(es) used to produce the care composition ingredient;
an assignor configured the decentral identifier and the environmental attribute(s);
a passport provider configured to provide the care composition ingredient passport or digital asset in association with the produced care composition ingredient to a decentral network, wherein the environmental attribute(s) associated with the care composition ingredient is made accessible to a consumer of the care composition ingredient through the care composition ingredient passport or digital asset.

In another aspect disclosed is a method, e.g. a computer-implemented method, for generating a care composition ingredient passport or digital asset associated with a care composition ingredient, wherein the care composition ingredient is produced from one or more input material(s) through one or more chemical process(es) of a chemical production network, wherein the one or more input material(s) and/or the one or more chemical process(es) are associated with environmental attribute(s), the method comprising:
providing a decentral identifier associated with the produced care composition ingredient and one or more environmental attribute(s) of the one or more input material(s) and/or the one or more chemical process(es) used to produce the care composition ingredient;
linking the decentral identifier and the environmental attribute(s);
providing the care composition ingredient passport or digital asset in association with the produced care composition ingredient to a decentral network, wherein the environmental attribute(s) associated with the care composition ingredient is made accessible to a user of the care composition ingredient through the care composition ingredient passport or digital asset.

In another aspect disclosed is a computer element, such as a computer readable storage medium, a computer program or a computer program product, comprising instructions, which when executed by a computing node or a computing system, direct the computing node or computing system to carry out the steps of the computer-implemented methods disclosed herein.

In another aspect disclosed is a computer element, such as a computer readable storage medium, a computer program or a computer program product, comprising instructions, which when executed by the apparatuses disclosed herein, direct the apparatuses to carry out steps the apparatuses disclosed herein are configured to execute.

In another aspect disclosed is a care composition ingredient associated with a care composition ingredient passport or a digital asset as produced according to the methods disclosed herein. In another aspect disclosed is a care composition ingredient associated with a care composition ingredient passport or digital asset as produced according to the systems disclosed herein.

In another aspect disclosed is a care composition ingredient associated with a care composition ingredient passport or digital asset, wherein the care composition ingredient is produced from one or more input material(s) through one or more chemical process(es) of a chemical production network, wherein the one or more input material(s) and/or the one or more chemical process(es) are associated with environmental attribute(s), wherein the care composition ingredient passport or digital asset includes a decentral identifier associated with the produced care composition ingredient and a link to environmental attribute(s) associated with one or more environmental attribute(s) of one or more input material(s) and/or one or more chemical process(es) used to produce the care composition ingredient.

In another aspect disclosed is a care composition ingredient passport or digital asset as generated according to the methods disclosed herein. In another aspect disclosed is a care composition ingredient passport or digital asset as generated according to the apparatuses disclosed herein.

In another aspect disclosed is a production system for producing a product from the care composition ingredient associated with the care composition ingredient passport or digital asset as provided according to the systems, apparatuses or methods disclosed herein. In another aspect disclosed is a production method for producing a product from the care composition ingredient associated with the care composition ingredient passport or digital asset as provided according to the systems, apparatuses or methods disclosed herein.

In another aspect disclosed is a use of the care composition ingredient associated with the care composition ingredient passport or digital asset as disclosed herein for producing a product from the care composition ingredient associated with the care composition ingredient passport or digital asset.

In another aspect disclosed is a use of the care composition ingredient passport or digital asset as disclosed herein for generating a product passport or digital asset associated with a product produced from the care composition ingredient associated with the care composition ingredient passport or digital asset. In another aspect disclosed is a method for using the digital asset generated according to the methods disclosed herein in the production of a product produced from the care composition ingredient associated with the care composition ingredient passport or digital asset.

In another aspect disclosed is a care composition ingredient associated with a digital asset including a decentral identifier associated with the care composition ingredient and linked to one or more environmental attribute(s) of the one or more input material(s) and/or the one or more chemical process(es) used to produce the care composition ingredient.

In another aspect disclosed is a use of the care composition ingredient associated with the care composition ingredient passport or digital asset for producing a product from the care composition ingredient and associating the care composition ingredient passport or digital asset with the product produced from the care composition ingredient. In another aspect disclosed is a use of the care composition ingredient associated with the care composition ingredient passport or digital asset for producing a product from the care composition ingredient and deriving a product passport or digital asset from the care composition ingredient passport or digital asset. In another aspect disclosed is a method for using the care composition ingredient associated with the digital asset for producing a product from the care composition ingredient as disclosed herein and deriving a digital asset associated with the product from the care composition ingredient passport or digital asset.

Any disclosure and embodiments described herein relate to the methods, the systems, chemical products, care composition ingredient, care composition ingredient passports or digital assets and the computer elements lined out above and below and vice versa. Advantageously, the benefits provided by any of the embodiments and examples equally apply to all other embodiments and examples.

### EMBODIMENTS

In the following, embodiments of the present disclosure will be outlined by ways of embodiments and/or example. It is to be understood that the present disclosure is not limited to said embodiments and/or examples. All terms and definitions used herein are understood broadly and have their general meaning.

Determining, generating includes initiating or causing to determine, generate. Providing includes “initiating or causing to access, determine, generate, send or receive”. “Initiating or causing to perform an action” includes any processing signal that triggers a computing node to perform the respective action.

The methods, the systems, care composition ingredients, care composition ingredient passports or digital assets and the computer elements disclosed herein provide an efficient, secure and robust way for sharing or exchanging environmental impact data across different participant nodes in value chains. In particular, by providing care composition ingredient specific data via the care composition ingredient passport or digital asset, environmental impacts can be shared and made transparent from the care composition ingredient to the product produced from such care composition ingredient. The care composition ingredient passport or digital asset enables secure data exchange, since data access can be controlled by the care composition ingredient provider, e.g. the entity providing the care composition ingredient. The exchanged data assets can be specific to the care composition ingredient as produced and tailored to the needs of the consumer of the care composition ingredient. This way an improved tracking and tracing of care composition ingredients can be achieved by securely providing environmental impact data in diverse and highly complex value chains. The environmental impact of care composition ingredient can hence be tracked leading to simpler, more efficient and sustainable handling of care composition ingredient by value chain participants.

Care composition ingredients associated with the care composition ingredients passport or digital asset are compounds used in compositions for various applications like fabric and home care, industrial and institutional cleaning, food and beverage processing, food service and kitchen hygiene, sanitation or vehicle and transportation care.

Non-limiting examples of care compositions where care composition ingredients are used are laundry detergent formulations, dish wash detergent formulations, hard surface cleaning compositions that may be used for cleaning various surfaces such as hard wood, tile, ceramic, plastic, leather, metal, glass, laundry cleaning formulations, laundry care products, laundry treatment products, laundry washing products.

Care composition ingredients according to this invention include, without being limited to, acids, alkali carriers,

Antimicrobials, preservatives, biocides, anti-oxidants, anti-tarnish agents, bleach components, brighteners, pH buffers, builders, co-builders, catalytic agents, chelating agents, crystal growth inhibitors, clay soil removal/anti-redeposition agents, color speckles, color care agents, corrosion inhibitors, defoamers, desiccants, dispersants, dye transfer inhibiting agents, dyes and hueing agents, dye clay conjugates, emollients, enzymes, enzyme stabilizers, fabric softeners, humectants, hydrotopes, grease cleaning agents, light stabilizer, malodor control agents, opacifiers, perfumes, fragrances, perfume delivery systems, photocatalytic systems, pigments, polymers, soil release agents, preformed peracids, processing aids, rheology modifiers, scrubbing particles, silver care, soaps, solubilizing agents, solvents, peroxide sources, structure elasticizing agents, suds boosters, suds suppressors, surfactants, surface care agents, UV light stabilizers, waxes, and whitening agents.

### Acids

Care composition ingredients according to this invention include acids. Acids include, without being limited to, methanesulfonic acid (e.g. Lutropur® MSA), formic acid (e.g. Luvipur® FM grades), and mixtures of dicarboxylic acids (e.g. Sokalan® DCS).

### Alkali Carriers

Care composition ingredients according to this invention include alkali carriers. Alkali carriers include alkali metal carbonates, alkali metal hydrogen carbonates, alkali metal metasilicates, alkali metal hydroxides, and amines. Preferred alkali metals are potassium and sodium. Amines include alkanolamines as for example triethanolamine.

### Antimicrobials, Preservatives, Biocides

Care composition ingredients according to this invention include antimicrobials, preservatives, and biocides like the following:
Benzylhemiformal, synonym: (Benzyloxy) methanol (CAS No. 14548-60-8);
(Ethylenedioxy)dimethanol, synonyms: Dascocide 9; (ethylenedioxy)dimethanol (reaction products of ethylene glycol with paraformaldehyde (EGForm)) (CAS. No. 3586-55-8);
α, α′, α″-trimethyl-1,3,5-triazine-1,3,5 (2H,4H,6H)-triethanol, synonyms: Tris (N-hydroxypropyl) hexahydrotriazine, hexahydro-1,3-5-tris(2-hydroxypropyl)-s-triazine (HPT, CAS No. 25254-50-6);
2,2-dibromo-2-cyanoacetamide (DBNPA, CAS No. 10222-01-2);
2,2′-dithiobis [N-methylbenzamide] (DTBMA, CAS No. 2527-58-4);
2-bromo-2-(bromomethyl) pentanedinitrile (DBDCB, CAS No. 35691-65-7);
2-Butanone, peroxide, synonym: 2-butanone-peroxide (CAS No. 1338-23-4);
2-butyl-benzo[d] isothiazol-3-one (BBIT, CAS No. 4299-07-4);
2-methyl-2H-isothiazol-3-one (MIT, CAS No 2682-20-4);
2-octyl-2H-isothiazol-3-one (OIT, CAS No. 26530-20-1);
5-Chloro-2-methyl-2H-isothiazol-3-one (CIT, CMIT, CAS No. 26172-55-4);
Mixture of 5-chloro-2-methyl-2H-isothiazol-3-one (CMIT, EINECS 247-500-7) and 2-methyl-2Hisothiazol-3-one (MIT, EINECS 220-239-6) (Mixture of CMIT/MIT, CAS No. 55965-84-9);
1,2-benzisothiazol-3 (2H)-one (BIT, CAS No. 2634-33-5);
3,3′-methylenebis [5-methyloxazolidine] (Oxazolidin/MBO, CAS No. 66204-44-2);
4,4-dimethyloxazolidine (CAS No. 51200-87-4);
7a-ethyldihydro-1H,3H,5H-oxazolo[3,4-c]oxazole (EDHO, CAS No. 7747-35-5);
Benzyl Alcohol (CAS No. 100-51-6);
Biphenyl-2-ol (CAS No. 90-43-7);
Biphenyl-2-ol, and its salts, o-phenylphenol, MEA-o-phenylphenate, potassium phenylphenate, sodium phenylphenate;
Sodium 2-biphenylate (CAS No. 132-27-4);
cis-1-(3-chloroallyl)-3,5,7-triaza-1-azoniaadamantane chloride (cis CTAC, CAS No. 51229-78-8);
Didecyldimethylammonium chloride (DDAC, CAS No. 68424-95-3 and CAS No. 7173-51-5);
Dodecylguanidine monohydrochloride (CAS No 13590-97-1);
Ethanol (CAS. No 64-17-5);
n-propanol (1-propanol, CAS No. 71-23-8)
Hexa-2,4-dienoic acid (Sorbic acid, CAS No. 110-44-1) and its salts, e.g. calcium sorbate, sodium sorbate
Potassium (E,E)-hexa-2,4-dienoate (Potassium Sorbate, CAS No. 24634-61-5);
Hydrogen peroxide (CAS No. 7722-84-1);
Lactic acid and its salts;
L-(+)-lactic acid (CAS No. 79-33-4);
2-methyl-1,2-benzothiazol-3 (2H)-one (MBIT, CAS No. 2527-66-4);
Methenamine 3-chloroallylochloride (CTAC, CAS No. 4080-31-3);
Monochloramine generated from ammonium carbamate and a chlorine source
N,N′-methylenebismorpholine (MBM, CAS No. 5625-90-1);
N-(3-aminopropyl)-N-dodecylpropane-1,3-diamine (Diamine, CAS No. 2372-82-9);
N-(trichloromethylthio) phthalimide (Folpet, CAS No. 133-07-3);
p-[(diodomethyl)sulphonyl]toluene (CAS No. 20018-09-1);
Peracetic acid (CAS No. 79-21-0);
polyhexamethylene biguanide hydrochloride (PHMB, CAS No 1802181-67-4), polyhexamethylene biguanide hydrochloride (PHMB, CAS No. 27083-27-8), e.g. poly (iminoimidocarbonyl)iminohexamethylene hydrochloride, poly (iminocarbonimidoyliminocarbonimidoylimino-1,6-hexanediyl), polyaminopropyl biguanide;
Pyridine-2-thiol 1-oxide, sodium salt (Sodium pyrithione, CAS No. 3811-73-2);
Pyrithione zinc (Zinc pyrithione, CAS No. 13463-41-7);
Reaction mass of titanium dioxide and silver chloride, silver chloride (CAS No. 7783-90-6);
Sodium Azide (CAS No. 26628-22-8);
Tetrahydro-1,3,4,6-tetrakis (hydroxymethyl) imidazo[4,5-d]imidazole-2,5 (1H,3H)-dione (TMAD, CAS No 5395-50-6);
Tetrakis (hydroxymethyl)phosphonium sulphate (2:1) (THPS, CAS No. 55566-30-8);
Salts of benzoic acid e.g. ammonium benzoate, calcium benzoate, magnesium benzoate, MEA-benzoate, potassium benzoate;
Esters of benzoic acid, e.g. butyl benzoate, ethyl benzoate, isobutyl benzoate, isopropyl benzoate, methyl benzoate, phenyl benzoate, propyl benzoate;
Benzoic acid and its sodium salt (CAS No 65-85-0, CAS No. 532-32-1);
Propionic acid and its salts, e.g. ammonium propionate, calcium propionate, magnesium propionate, potassium propionate, sodium propionate;
Salicylic acid and its salts, e.g. calcium salicylate, magnesium salicylate, MEA salicylate, sodium salicylate, potassium salicylate, TEA salicylate;
Inorganic sulphites and hydrogensulphites, e.g. sodium sulfite, ammonium sulfite, ammonium bisulfite, potassium sulfite, potassium hydrogene sulfite, sodium bisulfite, sodium metasulfite, potassium metasulfite, potassium metabisulfite;
Chlorobutanol (CAS No 57-15-8);
Butyl 4-hydroxybenzoate and its salts e.g. butylparaben, sodium butyl paraben, potassium butyl paraben;
Propyl 4-hydroxybenzoate and its salts, e.g. propyl paraben, sodium propyl paraben, potassium propyl paraben;
Isopropyl-4-hydroxybenzoic acid and its salts and esters.
Isobutyl-4-hydroxybenzoic acid and its salts and esters;
Benzyl-4-hydroxybenzoic acid and its salts and esters;
Pentyl-4-hydroxybenzoic acid and its salts and esters;
4-Hydroxybenzoic acid and its salts and esters, e.g. methyl paraben, ethyl paraben, potassium ethyl paraben, potassium paraben, potassium methyl paraben, sodium methyl paraben, sodium ethyl paraben, sodium paraben, calcium paraben, calcium methyl paraben, calcium ethyl paraben;
3-Acetyl-6-methylpyran-2,4 (3H)-dione and its salts, e.g. dehydroacetic acid, sodium dehydroacetic acid (Cas Nos 520-45-6, 4418-26-2, 16807-48-0);
3,3′-Dibromo-4,4′-hexamethylenedioxydibenzamidine and its salts (including isethionate), e.g. dibromohexamidine isethionate (CAS No. 93856-83-8);
Thiomersal (CAS No 54-64-8);
Phenylmercuric salts (including borate), e.g. phenyl mercuric acetate, phenyl mercuric benzoate (CAS Nos. 62-38-4 and 94-43-9);
Undec-10-enoic acid and its salts, e.g. undecylenic acid, potassium undecylenic acid, sodium undecylenic acid, calcium undecylenic acid, MEA-undecylenic acid, TEA-undecylenic acid;
5-Pyrimidinamine, 1,3-bis(2-ethylhexyl) hexahydro-5-methyl-, e.g. hexetidine (CAS No. 141-94-6);
1-(4-Chlorophenyl)-3-(3,4-dichlorophenyl) urea, e.g. triclocarban (CAS No 101-20-2);
Chlorocresol, e,g, p-chloro-m-cresol (CAS No. 59-50-7);
Chloroxylenol (CAS Nos 88-04-0, 1321-23-9);
N,N″-Methylenebis[N′-[3-(hydroxymethyl)-2,5-dioxoimidazolidin-4-yl]urea], synonym: imidazolidinyl urea (CAS No. 39236-46-9);
Methenamine (CAS No. 100-97-0);
Methenamine 3-chloroallylochloride, synonym: Quaternium 15 (CAS No 4080-31-3),
1-(4-Chlorophenoxy)-1-(imidazol-1-yl)-3,3-dimethylbutan-2-one, synonym: Climbazole (CAS No 38083-17-9);
1,3-Bis (hydroxymethyl)-5,5-dimethylimidazolidine-2,4-dione, synonym: DMDM hydantoin (CAS No 6440-58-0);
1-Hydroxy-4-methyl-6-(2,4,4-trimethylpentyl)-2 pyridon and its monoethanolamine salt, e.g. 1-hydroxy-4-methyl-6-(2,4,4-trimethylpentenyl)-2-pyridon, piroctone olamine (CAS Nos 50650-76-5, 68890-66-4);
2,2′-Methylenebis(6-bromo-4-chlorophenol), synonym: bromochorophene (CAS No 15435-29-7);
4-Isopropyl-m-cresol, synonym: o-cymen-5-ol (CAS No 3228-02-2);
2-Benzyl-4-chlorophenol, synonym: chlorophene (CAS No 120-32-1);
2-Chloroacetamide (CAS No 79-07-2);
N,N′-bis(4-chlorophenyl)-3, 12-diimino-2,4,11,13-tetraazatetradecanediamidine and its digluconate, diacetate and dihydrochloride, e.g. chlorohexidine, chlorhexidine digluconate, chlorohexidine diacetate, chlorhexidine dihydrochloride (CAS Nos 55-56-1, 56-95-1, 18472-51-0, 3697-42-5);
Alkyl (C12-C22)trimethyl ammonium bromide and chloride, e.g. behentrimonium chloriode, cetrimonium bromide, cetrimonium chloride, laurtrimonium bromide, laurtrimonium chloride, steartrimonium bromide, steartrimonium chloride (CAS Nos 17301-53-0, 57-09-0, 112-02-7, 1119-94-4, 112-00-5, 1120-02-1, 112-03-8);
4,4-Dimethyl-1,3-oxazolidine (CAS No 51200-87-4);
N-(Hydroxymethyl)-N-(dihydroxymethyl-1,3-dioxo-2,5-imidazolidinyl-4)-N′-(hydroxymethyl) urea, synonym: diazolidinyl urea (CAS No 78491-02-8);
Benzenecarboximidamide, 4,4′-(1,6-hexanediylbis(oxy))bis-, and its salts (including isothionate and p-hydroxybenzoate), e.g. hexamidine, hexamidine diisethionate, hexamidine paraben (CAS Nos 3811-75-4, 659-40-5, 93841-83-9);
5-Ethyl-3,7-dioxa-1-azabicyclo[3.3.0] octane, synonym: 7-ethylbicyclooxazolidine (CAS No 7747-35-5); 5
3-(p-Chlorophenoxy)-propane-1,2-diol, synonym: chlorophenesin (CAS No 104-29-0);
Sodium hydroxymethylamino acetate, synonym: sodium N-(hydroxymethyl)glycinate, sodium hydroxymethylglycinate (CAS No 70161-44-3);
Benzenemethanaminium, N,N-dimethyl-N-[2-[2-[4-(1,1,3,3,10 tetramethylbutyl) phenoxy]ethoxy]ethyl]-, chloride, synonym: benzethonium chloride CAS No 121-54-0);
Benzalkonium chloride, bromide and saccharinate, e.g. benzalkonium chloride, benzalkonium bromide, benzalkonium saccharinate (CAS Nos 8001-54-5, 63449-41-2, 91080-29-4, 68989-01-5, 68424-85-1, 68391-01-5, 61789-71-7, 85409-22-9);
Methanol, (phenylmethoxy), synonym: benzylhemiformal (CAS No 14548-60-8);
3-Iodo-2-propynylbutylcarbamate (IPBC, CAS No 55406-53-6)
Ethyl Lauroyl Arginate HCl (CAS No 60372-77-2);
1,2,3-Propanetricarboxylic acid, 2-hydroxy-, monohydrate and 1,2,3-Propanetricarboxylic acid, 2-hydroxy-silver (1+) salt, monohydrate, INCI: citric acid (and) silver citrate;
Tetrahydro-3,5-dimethyl-1,3,5-thiadia-zine-2-thione (further names: 3,5-dimethyl-1,3-5-thiadiazinane-2-thione, Protectol® DZ, Protectol® DZ P, Dazomet, CAS No. 533-74-4);
2,4-dichlorobenzyl alcohol (CAS-No. 1777-82-8, further names: dichlorobenzyl alcohol, 2,4-dichloro-benzenemethanol, (2,4-dichloro-phenyl)-methanol, DCBA, Protectol® DA);
1-propanol (CAS-No. 71-23-8, further names: n-propanol, propan-1-ol, n-propyl alcohol, Protectol® NP S);
5-bromo-5-nitro-1,3-dioxane (CAS-No. 30007-47-7, further names: 5-bromo-5-nitro-m-dioxane, Bronidox®);
2-bromo-2-nitropropane-1,3-diol (CAS-No. 52-51-7, further names: 2-bromo-2-nitro-1,3-propanediol, Bronopol®, Protectol® BN, Myacide AS);
Glutaraldehyde (CAS-No. 111-30-8, further names: 1-5-pentandial, pentane-1,5-dial, glutaral, glutardialdehyde, Protectol® GA, Protectol® GA 50, Myacide® GA);
Glyoxal (CAS No. 107-22-2; further names: ethandial, oxylaldehyde, 1,2-ethandial, Protectol® GL);
2,4,4′-trichloro-2′-hydroxydiphenyl ether (CAS No. 3380-34-5, further names: triclosan, Irgasan® DP 300, Irgacare® MP, TCS);
4,4′-dichloro 2-hydroxydiphenyl ether (CAS-No. 3380-30-1), further names: 5-chloro-2-(4-chlorophenoxy) phenol, Diclosan, DCPP, which is commercially available as a solution of 30 wt % of 4,4′-dichloro 2-hydroxydiphenyl ether in 1,2 propyleneglycol under the trade name Tinosan® HP 100 (BASF);
2-Phenoxyethanol (CAS-no. 122-99-6, further names: Phenoxyethanol, Methylphenylglycol, Phenoxetol, ethylene glycol phenyl ether, Ethylene glycol monophenyl ether, Protectol® PE);
Phenoxypropanol (CAS-No. 770-35-4, CAS No 4169-04-4, propylene glycol phenyl ether, phenoxyisopropanol 1-phenoxy-2-propanol, 2-phenoxy-1-propanol);
Glucoprotamine (CAS-No. 164907-72-6, chemical description: reaction product of glutamic acid and alkylpropylenediamine, further names: Glucoprotamine 50);
Cyclohexyl hydroxyl diazenium-1-oxide, potassium salt (CAS No. 66603-10-9, further names: N-cyclohexyl-diazenium dioxide, Potassium HDO, Xyligene, Protectol® KD);
Formic acid (CAS-No. 64-18-6, further names: methanoic acid, Protectol® FM, Protectol® FM 75, Protectol® FM 85, Protectol® FM 99, Lutensol® FM) and its salts, e.g. sodium formiate (CAS No 141-53-7);
Performic acid and its salts;
anorganic silver complexes such as silver zeolites and silver glass compounds (e.g. Irgaguard® B5000, Irgaguard® B6000, Irgaguard® B7000) and others described in WO-A-99/18790, EP1041879B1;
1,3,5-Tris-(2-hydroxyethyl)-hexahydro-1,3,5-triazin (CAS-No. 4719-04-4, further names: Hexyhydrotriazine, Tris(hydroethyl)-hexyhydrotriazin, hexyhydro-1,3,5-tris(2-hydroxyethyl)-striazine, 2,2′,2″-(hexahydro-1,3,5-triazine-1,3,5-triyl)triethanol, Protectol® HT).

### Anti-Oxidants

Care composition ingredients according to this invention include anti-oxidants. Anti-oxidants include, without being limited to, Didodecyl 3,3′-thiodipropionate (e.g. Tinogard® DA), Octadecyl-3-(3,5-di-tert-butyl-4-hydroxyphenyl)-propionate (e.g. Tinogard® TS), Pentaerythritol tetrakis(3-(3,5-di-tert-butyl-4-hydroxyphenyl) propionate) (e.g. Tinogard® TT).

### Bleach Components

Care composition ingredients according to this invention include bleach components. Bleach components include bleaching catalysts, photobleaches, bleach activators, hydrogen peroxide, sources of hydrogen peroxide, and pre-formed peracids.

Bleach components include pre-formed peracids. Preformed peracids include, but are not limited to, compounds selected from the group consisting of pre-formed peroxyacids or salts thereof, typically either a peroxycarboxylic acid or salt thereof, or a peroxysulphonic acid or salt thereof.

The pre-formed peroxyacid or salt thereof can be a peroxycarboxylic acid or salt thereof, typically having a chemical structure corresponding to the following chemical formula

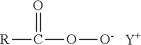

wherein R is selected from alkyl, aralkyl, cycloalkyl, aryl or heterocyclic groups; the R group can be linear or branched, substituted or unsubstituted; and Y is any suitable counter-ion that achieves electric charge neutrality, preferably Y is selected from hydrogen, sodium or potassium. Preferably, R is a linear or branched, substituted or unsubstituted C₆-C₉ alkyl. Preferably, the peroxyacid or salt thereof is selected from peroxyhexanoic acid, peroxyheptanoic acid, peroxyoctanoic acid, peroxynonanoic acid, peroxydecanoic acid, any salt thereof, or any combination thereof. Particularly preferred peroxyacids are phthalimido-peroxy-alkanoic acids, ε-phthalimido peroxy hexanoic acid (PAP). Preferably, the peroxyacid or salt thereof has a melting point in the range of from 30° C. to 60° C.

The pre-formed peroxyacid or salt thereof can also be a peroxysulphonic acid or salt thereof, typically having a chemical structure corresponding to the following chemical formula:

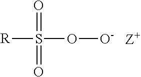

wherein R is selected from alkyl, aralkyl, cycloalkyl, aryl or heterocyclic groups; the R group can be linear or branched, substituted or unsubstituted; and Z is any suitable counter-ion that achieves electric charge neutrality, preferably Z is selected from hydrogen, sodium or potassium. Preferably R is a linear or branched, substituted or unsubstituted C₆-C₉ alkyl. Sources of hydrogen peroxide include inorganic perhydrate salts, including alkali metal salts such as sodium salts of perborate (usually mono- or tetra-hydrate), percarbonate, persulphate, perphosphate, and persilicate salts.

The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable bleach activators are those having R—(O)-L, wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or less than 4 carbon atoms; and L is leaving group. Examples of suitable leaving groups are benzoic acid and derivatives thereof-especially benzene sulphonate. Bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED), sodium 4-[(3,5,5-trimethylhexanoyl)oxy] benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), the family of bleach activators disclosed in EP624154, acetyl triethyl citrate (ATC), triacetin, tetraacetylmethylene diamine, tetraacetylglycoluril, tetraacetylhexylene diamine, acylated phenolsulfonates such as n-nonanoyl- or isononanoyloxybenzene sulfonates, N-methylmorpholinium-acetonitrile salts (“MMA salts”), trimethylammonium acetonitrile salts, N-acylimides such as N-nonanoylsuccinimide, 1,5-diacetyl-2,2-dioxohexahydro-1,3,5-triazine (“DADHT”), and nitrile quats (trimethylammonium acetonitrile salts).

Diacyl peroxides (DAP) include those selected from diacyl peroxides of the general formula: R¹—C (O)—OO—(O)C—R², in which R¹ represents a C₆-C₁₈ alkyl, preferably C₆-C₁₂ alkyl group containing a linear chain of at least 5 carbon atoms and optionally containing one or more substituents (e.g., —N+(CH₃)₃, —COOH or —CN) and/or one or more interrupting moieties (e.g., —CONH— or —CH—) interpolated between adjacent carbon atoms of the alkyl radical, and R² represents an aliphatic group compatible with a peroxide moiety, such that R¹ and R² together contain a total of 8 to 30 carbon atoms. In one preferred aspect R¹ and R² are linear unsubstituted C₆-C₁₂ alkyl chains. Most preferably R¹ and R² are identical. Diacyl peroxides, in which both R¹ and R² are C₆-C₁₂ alkyl groups, are particularly preferred. Preferably, at least one of, most preferably only one of, the R groups (R¹ or R²), does not contain branching or pendant rings in the alpha position, or preferably neither in the alpha nor beta positions or most preferably in none of the alpha or beta or gamma positions. In one further preferred embodiment the DAP may be asymmetric, such that preferably the hydrolysis of R¹ acyl group is rapid to generate peracid, but the hydrolysis of R² acyl group is slow.

The tetraacyl peroxide bleaching species is preferably selected from tetraacyl peroxides of the general formula: R³—C(O)—OO—C(O)—(CH₂)n-C(O)—OO—C(O)—R³, in which R³ represents a C₁-C₉ alkyl, or C₃-C₇ group and n represents an integer from 2 to 12, or 4 to 10 inclusive.

Bleach components further include bleach catalysts. capable of accepting an oxygen atom from a peroxyacid and/or salt thereof and transferring the oxygen atom to an oxidizeable substrate. Suitable bleach catalysts include but are not limited to: iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof. Iminium cations and polyions include, but are not limited to, N-methyl-3,4-dihydroisoquinolinium tetrafluoroborate, prepared as described in Tetrahedron 49 (2): 423-38 (1992) (e.g., compound 4, p. 433); N-methyl-3,4-dihydroisoquinolinium p-toluene sulphonate, prepared as described in U.S. Pat. No. 5,360,569 (e.g., Column 11, Example 1); and N-octyl-3,4-dihydroisoquinolinium p-toluene sulphonate, prepared as described in U.S. Pat. No. 5,360,568 (e.g., Column 10, Ex. 3).

Iminium zwitterions include, but are not limited to, N-(3-sulfopropyl)-3,4-dihydroisoquinolinium, inner salt, prepared as described in U.S. Pat. No. 5,576,282 (e.g., Column 31, Ex. II); N-[2-(sulphooxy) dodecyl]-3,4-dihydroisoquinolinium, inner salt, prepared as described in U.S. Pat. No. 5,817,614 (e.g., Column 32, Ex. V); 2-[3-[(2-ethylhexyl)oxy]-2-(sulphooxy) propyl]-3,4-dihydroisoquinolinium, inner salt, prepared as described in WO 2005/047264 (e.g., p. 18, Ex. 8), and 2-[3-[(2-butyloctyl)oxy]-2-(sulphooxy) propyl]-3,4-dihydroisoquinolinium, inner salt.

Modified amine oxygen transfer catalysts include, but are not limited to, 1,2,3,4-tetrahydro-2-methyl-1-isoquinolinol, which can be made according to the procedures described in Tetrahedron Letters 28 (48): 6061-6064 (1987).

Modified amine oxide oxygen transfer catalysts include, but are not limited to, sodium 1-hydroxy-N-oxy-N-[2-(sulphooxy) decyl]-1,2,3,4-tetrahydroisoquinoline.

N-sulphonyl imine oxygen transfer catalysts include, but are not limited to, 3-methyl-1,2-benzisothiazole-1,1-dioxide, prepared according to the procedure described in the Journal of Organic Chemistry 55 (4): 1254-61 (1990).

N-phosphonyl imine oxygen transfer catalysts include, but are not limited to, [R-(E)]-N-[(2-chloro-5-nitrophenyl)methylene]-P-phenyl-P-(2,4,6-trimethylphenyl)-phosphinic amide, which can be made according to the procedures described in the Journal of the Chemical Society, Chemical Communications (22): 2569-2570 (1994).

N-acyl imine oxygen transfer catalysts include, but are not limited to, [N (E)]-N-(phenylmethylene) acetamide, which can be made according to the procedures described in the Polish Journal of Chemistry 77 (5): 577-590 (2003).

Thiadiazole dioxide oxygen transfer catalysts include but are not limited to, 3-methyl-4-phenyl-1,2,5-thiadiazole-1,1-dioxide, which can be made according to the procedures described in U.S. Pat. No. 5,753,599 (Column 9, Ex. 2).

Perfluoroimine oxygen transfer catalysts include, but are not limited to, (Z)-2,2,3,3,4,4,4-heptafluoro-N-(nonafluorobutyl) butanimidoyl fluoride, which can be made according to the procedures described in Tetrahedron Letters 35 (34): 6329-6330 (1994).

Cyclic sugar ketone oxygen transfer catalysts include, but are not limited to, 1,2:4,5-di-O-isopropylidene-D-erythro-2,3-hexodiuro-2,6-pyranose as prepared in U.S. Pat. No. 6,649,085 (Column 12, Ex. 1).

Photobleaches include, e.g., sulfonated zinc phthalocyanine sulfonated aluminium phthalocyanines and xanthene dyes.

### Brighteners

Care composition ingredients according to this invention include brighteners. Brighteners include fluorescent brighteners like C.I. fluorescent brightener 260 in alpha-crystalline form having the following structure:

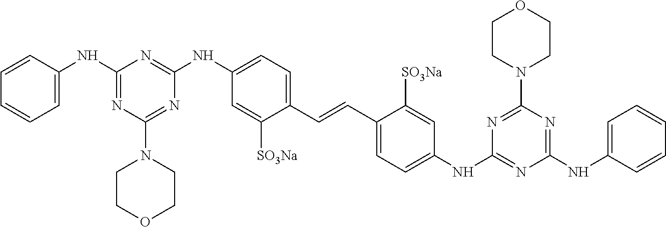

Brighteners further include derivatives of stilbene, pyrazoline, coumarin, carboxylic acid, methinecyanines, dibenzothiophene-5,5-dioxide, azoles, 5- and 6-membered-ring heterocycles, and other miscellaneous agents. Examples of such brighteners are disclosed in “The Production and Application of Fluorescent Brightening Agents”, M. Zahradnik, Published by John Wiley & Sons, New York (1982).

### Builders/Co-Builders

Care composition ingredients according to this invention include builders and co-builders. Builders and co-builders comprise complexing agents, ion exchange compounds, and precipitating agents. Builders include citrates, phosphates, silicates, carbonates, phosphonates, polyphosphonates, amino carboxylates, polycarboxylates, polysulfonates.

Citrates include mono- and dialkali metal salts, in particular mono- and preferably the trisodium salt of citric acid, ammonium or substituted ammonium salts of citric acid as well as citric acid. Citrates can be used as the anhydrous compound or as the hydrate, for example as sodium citrate dihydrate.

Phosphates include sodium metaphosphate, sodium orthophosphate, sodium hydrogenphosphate, sodium pyrophosphate and polyphosphates such as sodium tripolyphosphate.

Silicates include sodium disilicate and sodium metasilicate, aluminosilicates such as sodium aluminosilicates like zeolith A (i.e. Na₁₂(AIO₂)₁₂(SiO₂)₁₂*27H₂O), and sheet silicates, in particular those of the formula alpha-Na₂Si₂O₅, beta-Na₂Si₂O₅, and delta-Na₂Si₂O₅.

Phosphates include sodium metaphosphate, sodium orthophosphate, sodium hydrogenphosphate, sodium pyrophosphate and polyphosphates such as sodium tripolyphosphate.

Carbonates include alkali metal carbonates and alkali metal hydrogen carbonates, preferably sodium salts, particularly preferred Na₂CO₃.

Phosphonates include 2-phosphinobutane-1,2,4-tricarboxylic acid (PBTC); ethylenediaminetetra(methylenephosphonic acid) (EDTMPA); 1-hydroxyethane-1,1-diphosphonic acid (HEDP), CH₂C(OH)[PO(OH)₂]₂; aminotris(methylenephosphonic acid) (ATMP), N[CH₂PO(OH)₂]₃; aminotris(methylenephosphonate), sodium salt (ATMP), N[CH₂PO(ONa)₂]₃; 2-hydroxyethyliminobis(methylenephosphonic acid), HOCH₂CH₂N[CH₂PO(OH)₂]₂; diethylenetriaminepenta (methylenephosphonic acid) (DTPMP), (HO)₂POCH₂N[CH₂CH₂N[CH₂PO(OH)₂]₂]₂; diethylenetriaminepenta (methylenephosphonate), sodium salt, C₉H₍₂₈₋ₓ₎N₃NaₓO₁₅P₅ (x=7); hexamethylenediamine (tetramethylenephosphonate), potassium salt, C₁₀H₍₂₈₋ₓ₎N₂Kₓ0₁₂P₄ (x=6); and bis(hexamethylene)triamine(pentamethylenephosphonic acid), (HO₂)POCH₂N[(CH₂)₂N[CH₂PO(OH)₂]₂]₂.

Further builders are a-hydroxypropionic acid and oxidized starch.

Amino carboxylates and polycarboxylates include nitrilotriacetates, ethylene diamine tetraacetate, diethylene triamine pentaacetate, triethylene tetraamine hexaacetate, propylene diamines tetraacetic acid, ethanol-diglycines, methylglycine diacetate, and glutamine diacetate. The terms “amino carboxylates” and “polycarboxylates” also include their respective non-substituted or substituted ammonium salts and the alkali metal salts such as the sodium salts, in particular the fully neutralized compound. Polycarboxylates include non-polymeric polycarboxylates such as succinic acid, C₂-C₁₆-alkyl disuccinates, C₂-C₁₆-alkenyl disuccinates, ethylene diamine N,N′-disuccinic acid, tartaric acid diacetate, alkali metal malonates, tartaric acid monoacetate, propanetricarboxylic acid, bu-tanetetracarboxylic acid and cyclopentanetetracarboxylic acid. Oligomeric or polymeric polycarboxylates are for example polyaspartic acid and alkali metal salts of (meth)acrylic acid homopolymers or (meth)acrylic acid copolymers. Suitable comonomers include monoethylenically unsaturated dicarboxylic acids such as maleic acid, fumaric acid, maleic anhydride, itaconic acid and citraconic acid. Polycarboxylates include polyacrylic acid, which preferably has a weight-average molecular weight Mw in the range from 2000 to 40000 g/mol, preferably 2000 to 10 000 g/mol, more preferably 3000 to 8000 g/mol. Further copolymeric polycarboxylates include those of acrylic acid with methacrylic acid and of acrylic acid or methacrylic acid with maleic acid and/or fumaric acid.

Further polycarboxylate builders are copolymers of at least one monomer from the group consisting of monoethylenically unsaturated C3-C10-mono- or C4-C10-dicarboxylic acids or anhydrides thereof, such as maleic acid, maleic anhydride, acrylic acid, methacrylic acid, fumaric acid, itaconic acid and citraconic acid, with at least one hydrophilically or hydrophobically modified co-monomer as listed below.

Suitable hydrophobic co-monomers include isobutene, diisobutene, butene, pentene, hexene and styrene, olefins with ten or more carbon atoms or mixtures thereof, such as, for example, 1-decene, 1-dodecene, 1-tetradecene, 1-hexadecene, 1-octadecene, 1-eicosene, 1-docosene, 1-tetracosene and 1-hexacosene, C22-α-olefin, a mixture of C20-C24-α-olefins and polyisobutene having on average 12 to 100 carbon atoms per molecule.

Suitable hydrophilic co-monomers are monomers with sulfonate or phosphonate groups, nonionic monomers with hydroxyl function or alkylene oxide groups. Such monomers include allyl alcohol, isoprenol, methoxypolyethylene glycol (meth)acrylate, methoxypolypropylene glycol (meth)acrylate, methoxypolybutylene glycol (meth)acrylate, methoxypoly(propylene oxide-coethylene oxide) (meth)acrylate, ethoxypolyethylene glycol (meth)acrylate, ethoxypolypropylene glycol (meth)acrylate, ethoxypolybutylene glycol (meth)acrylate and ethoxypoly(propylene oxide-co-ethylene oxide) (meth)acrylate. Polyalkylene glycols comprise 3 to 50, in particular 5 to 40 and especially 10 to 30 alkylene oxide units per molecule.

Sulfonic-acid-group-containing monomers are 1-acrylamido-1-propanesulfonic acid, 2-acrylamido-2-propanesulfonic acid, 2-acrylamido-2-methylpropanesulfonic acid, 2-methacrylamido-2-methylpropanesulfonic acid, 3-methacrylamido-2-hydroxypropanesulfonic acid, allylsulfonic acid, methallylsulfonic acid, al-lyloxybenzenesulfonic acid, methallyloxybenzenesulfonic acid, 2-hydroxy-3-(2-propenyloxy) propanesulfonic acid, 2-methyl-2-propene-1-sulfonic acid, styrenesulfonic acid, vinylsulfonic acid, 3-sulfopropyl acrylate, 2-sulfoethyl methacrylate, 3-sulfopropyl methacrylate, sulfomethacrylamide, sulfomethylmethacrylamide, and salts of said acids, such as sodium, potassium or ammonium salts thereof.

Preferred phosphonate-group-containing monomers are vinylphosphonic acid and its salts. Builders further include amphoteric polymers.

Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), etheylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diylbis(phosphonic acid) (HEDP), ethylenediaminetetrakis(methylene)tetrakis(phosphonic acid) (EDTMPA), diethylenetriaminepentakis (methylene) pentakis (phosphonic acid) (DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MI DA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TLIDA) and sulfomethyl-N,N-diacetic acid (SMDA), N-(hydroxyethyl)-ethylidenediaminetriacetate (HEDTA), diethanolglycine (DEG), Diethylenetriamine Penta (Methylene Phosphonic acid) (DTPMP), aminotris(methylenephosphonic acid) (ATMP), and combinations and salts thereof. Further exemplary builders and/or co-builders are described in, e.g., WO 2009/102854, U.S. Pat. No. 5,977,053.

### Chelating Agents and Crystal Growth Inhibitors

Care composition ingredients according to this invention include chelating agent and crystal growth inhibitors. Chelating agent and crystal growth inhibitors include copper, iron and/or manganese chelating agents and mixtures thereof, DTPA (Diethylene triamine pentaacetic acid), HEDP (Hydroxyethane diphosphonic acid), DTPMP (Diethylene triamine penta (methylene phosphonic acid)), 1,2-Dihydroxybenzene-3,5-disulfonic acid disodium salt hydrate, ethylenediamine, diethylene triamine, ethylenediaminedisuccinic acid (EDDS), N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), carboxymethyl inulin and 2-Phosphonobutane 1,2,4-tricarboxylic acid (Bayhibit® AM) and derivatives thereof.

Examples of chelating agents are given in the Table “Chelating Agents” below:

### Table “Chelating Agents”

| Trade name | Chemical constitution |
|---|---|
| Trilon ® B | Tetrasodium salt of EDTA |
| Trilon ® BD | Disodium salt of Ethylenediaminetetraacetic acid |
| | (EDTA) |
| Trilon ® BS | Ethylenediaminetetraacetic acid |
| Trilon ® BX | Tetrasodium salt of EDTA |
| Trilon ® M | Trisodium salt of Methylglycinediacetic acid (MGDA) |
| Trilon ® M Max | Trisodium salt of Methylglycinediacetic acid (MGDA) |
| Trilon ® | Preparation based on Trisodium salt of MGDA |
| Ultimate | |

### Color Care Agents

Care composition ingredients according to this invention include color care agents. Color care agents include polymers like Polyvinylpyrrolidone (e.g. Sokalan® HP 53 and Sokalan® HP 165) and Vinylpyrrolidone/Vinylimidazole copolymers (e.g. Sokalan® HP 56 and Sokalan® HP 66).

### Corrosion Inhibitors

Care composition ingredients according to this invention include corrosion inhibitors. Corrosion inhibitors include triazoles, in particular benzotriazoles, bisbenzotriazoles, aminotri-azoles, alkylaminotriazoles, also phenol derivatives such as, for example, hydroquinone, pyro-catechol, hydroxyhydroquinone, gallic acid, phloroglucinol or pyrogallol.

Corrosion inhibitors include aliphatic dicarboxylic acid monoalkylamides like Korantin® MAT and Oleylsarcosine acid like Korantin® TC-SH.

### Dispersants

Care composition ingredients according to this invention include dispersants. Dispersants include water-soluble organic materials like the homo- or co-polymeric acids or their salts, in which the polycarboxylic acid comprises at least two carboxyl radicals separated from each other by not more than two carbon atoms.

Dispersants include the compounds listed in the table “Dispersants” below:

### Table “Dispersants”

Sokalan® type products are used as scale inhibitors or dispersing agents for stabilisation of dispersions in various fields of application.

| Trade name | Chemical constitution |
|---|---|
| Sokalan ® CP 10 | Polyacrylic acid, modified, sodium salt |
| Sokalan ® ® CP 10 S | Polyacrylic acid |
| Sokalan ® CP 12 | Maleic acid/acrylic acid copolymer |
| Sokalan ® CP 2695 | Polyacrylic acid, fully neutralized, sodium salt |
| Sokalan ® CP 13 | Polyacrylic acid, modified |
| Sokalan ® CP 45 | Maleic acid/acrylic acid copolymer, sodium salt |
| Sokalan ® CP 5 | Maleic acid/acrylic acid copolymer, sodium salt |
| Sokalan ® CP 50 | Polycarboxylate, modified, sodium salt |
| Sokalan ® CP 7 | Maleic acid/acrylic acid copolymer, sodium salt |
| Sokalan ® CP 9 | Maleic acid/olefin copolymer, sodium salt |
| Sokalan ® HP 20 | Multifunctional polyethylene imine |
| Sokalan ® HP 25 | Polycarboxylate, modified |
| Sokalan ® HP 96 | Quaternated, ethoxylated Hexamethylene |
| | diamine |
| Sokalan ® PA 15 | Polyacrylic acid, sodium salt |
| Sokalan ® PA 25 | Polycarboxylate, modified |
| Sokalan ® PA 30 | Polyacrylic acid, sodium salt |

### Dyes and Hueing Agents

Fabric hueing agents include dyes, dye-clay conjugates, and pigments.

Dyes include small molecule dyes and polymeric dyes. Small molecule dyes include small molecule dyes selected from the group consisting of dyes falling into the Color Index (C.I.) classifications of Direct Blue, Direct Red, Direct Violet, Acid Blue, Acid Red, Acid Violet, Basic Blue, Basic Violet and Basic Red.

Small molecule dyes also include small molecule dyes selected from the group consisting of Color Index (Society of Dyers and Colorists, Bradford, UK) numbers Direct Violet 9, Direct Violet 35, Direct Violet 48, Direct Violet 51, Direct Violet 66, Direct Violet 99, Direct Blue 1, Direct Blue 71, Direct Blue 80, Direct Blue 279, Acid Red 17, Acid Red 73, Acid Red 88, Acid Red 150, Acid Violet 15, Acid Violet 17, Acid Violet 24, Acid Violet 43, Acid Red 52, Acid Violet 49, Acid Violet 50, Acid Blue 15, Acid Blue 17, Acid Blue 25, Acid Blue 29, Acid Blue 40, Acid Blue 45, Acid Blue 75, Acid Blue 80, Acid Blue 83, Acid Blue 90 and Acid Blue 113, Acid Black 1, Basic Violet 1, Basic Violet 3, Basic Violet 4, Basic Violet 10, Basic Violet 35, Basic Blue 3, Basic Blue 16, Basic Blue 22, Basic Blue 47, Basic Blue 66, Basic Blue 75, Basic Blue 159, dyes selected from the group consisting of Color Index (Society of Dyers and Colorists, Bradford, UK) numbers Acid Violet 17, Acid Violet 43, Acid Red 52, Acid Red 73, Acid Red 88, Acid Red 150, Acid Blue 25, Acid Blue 29, Acid Blue 45, Acid Blue 113, Acid Black 1, Direct Blue 1, Direct Blue 71, Direct Violet 51, small molecule dyes further include small molecule dyes selected from the group consisting of Color Index (Society of Dyers and Colorists, Bradford, UK) numbers Acid Violet 17, Direct Blue 71, Direct Violet 51, Direct Blue 1, Acid Red 88, Acid Red 150, Acid Blue 29, Acid Blue 113.

Polymeric dyes include polymeric dyes selected from the group consisting of polymers containing conjugated chromogens (dye-polymer conjugates) and polymers with chromogens copolymerized into the backbone of the polymer and mixtures thereof.

Polymeric dyes include polymeric dyes selected from the group consisting of fabric-substantive colorants sold under the name of Liquitint® (Milliken), dye-polymer conjugates formed from at least one reactive dye and a polymer selected from the group consisting of polymers comprising a moiety selected from the group consisting of a hydroxyl moiety, a primary amine moiety, a secondary amine moiety, a thiol moiety and mixtures thereof.

Polymeric dyes also include polymeric dyes selected from the group consisting of Liquitint® Violet CT, carboxymethyl cellulose (CMC) conjugated with a reactive blue, reactive violet or reactive red dye such as CMC conjugated with C.I. Reactive Blue 19, sold by Megazyme, Wicklow, Ireland under the product name AZO-CM-CELLULOSE, product code S-ACMC, alkoxylated triphenyl-methane polymeric colorants, alkoxylated thiophene polymeric colorants, and mixtures thereof.

Hueing agents include the whitening agents as described in WO 2022/103725, pages 24-25, structures (I), (II), and (III) and Table 1.

Further examples of whitening agents are Distyryl biphenyl derivatives like the commercially available Tinopal® CBS grades.

### Dye Clay Conjugates

Care composition ingredients according to this invention include dye clay conjugates. Dye clay conjugates include dye clay conjugates selected from the group comprising at least one cationic/basic dye and a smectite clay, and mixtures thereof. Dye clay conjugates include C.I. Basic Yellow 1 through 108, C.I. Basic Orange 1 through 69, C.I. Basic Red 1 through 118, C.I. Basic Violet 1 through 51, C.I. Basic Blue 1 through 164, C.I. Basic Green 1 through 14, C.I. Basic Brown 1 through 23, CI Basic Black 1 through 11, Montmorillonite clay, Hectorite clay, Saponite clay, Montmorillonite Basic Blue B7 C.I. 42595 conjugate, Montmorillonite Basic Blue B9 C.I. 52015 conjugate, Montmorillonite Basic Violet V3 C.I. 42555 conjugate, Montmorillonite Basic Green G1 C.I. 42040 conjugate, Montmorillonite Basic Red R1 C.I. 45160 conjugate, Montmorillonite C.I. Basic Black 2 conjugate, Hectorite Basic Blue B7 C.I. 42595 conjugate, Hectorite Basic Blue B9 C.I. 52015 conjugate, Hectorite Basic Violet V3 C.I. 42555 conjugate, Hectorite Basic Green G1 C.I. 42040 conjugate, Hectorite Basic Red R1 C.I. 45160 conjugate, Hectorite C.I. Basic Black 2 conjugate, Saponite Basic Blue B7 C.I. 42595 conjugate, Saponite Basic Blue B9 C.I. 52015 conjugate, Saponite Basic Violet V3 C.I. 42555 conjugate, Saponite Basic Green G1 C.I. 42040 conjugate, Saponite Basic Red R1 C.I. 45160 conjugate, Saponite C.I. Basic Black 2 conjugate

### Enzymes

Care composition ingredients according to this invention include enzymes. Enzymes are preferably selected from proteases, lipases, amylases, mannanases, cellulases, hydrolases, hemicellulases, phospholipases, esterases, pectinases, lactases, pectate lyases, cutinases, phosphodiesterases (PDEs), deoxyribonucleases (DNases), xylanases, oxicoreductases, dispersins, peroxidases, and combinations of at least two of the foregoing enzymes.

### Proteases

Proteases are active proteins exerting “protease activity” or “proteolytic activity”. Proteolytic activity is related to the rate of degradation of protein by a protease or proteolytic enzyme in a defined course of time.

Preferably, the protease is selected from proteases with at least 40 to 100% identity to the full-length polypeptide sequence of any of SEQ ID NO: 10-14. In one embodiment, the protease comprises an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the full length polypeptide sequence shown in SEQ ID NO: 10, 11, 12,13, or 14, preferably SEQ ID NO: 10.

Preferably, the protease comprises an amino acid sequence having at least 80%, at least 85%, at least 90%, at least 91%, at least 92%, at least 93%, at least 94%, at least 95%, at least 96%, at least 97%, at least 98%, or at least 99%, but below 100% sequence identity with SEQ ID NO: 10 and further comprises amino acid substitutions in one or more of the following positions 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 199, 205, 206, 217, 218, 222, 224, 232, 235, 236, 245, 248, 252 and 274 (according to the BPN′ numbering) and which has proteolytic activity. In one embodiment, such a protease is not mutated at positions Asp32, His64 and Ser221 (according to BPN′ numbering). Preferably, the protease comprises an amino acid sequence having at least 80%, at least 85%, at least 90%, at least 91%, at least 92%, at least 93%, at least 94%, at least 95%, at least 96%, at least 97%, at least 98%, or at least 99%, but below 100% sequence identity with SEQ ID NO: 10 and is further characterized by having amino acid glutamic acid (E), or aspartic acid (D), or asparagine (N), or glutamine (Q), or alanine (A), or glycine (G), or serine(S), preferably glutamic acid (E), at position 101 (according to BPN′ numbering) and has proteolytic activity. Mostly preferred is a protease that has at least 80%, but below 100% sequence identity with SEQ ID NO: 10 and is characterized by having amino acid glutamic acid (E) at position 101 (according to BPN′ numbering) and that has proteolytic activity. The protease may comprise an amino acid substitution at position 101, such as R101E alone or in combination with one or more substitutions at positions 3, 4, 9, 15, 24, 27, 33, 36, 57, 68, 76, 77, 87, 95, 96, 97, 98, 99, 100, 102, 103, 104, 106, 118, 120, 123, 128, 129, 130, 131, 154, 160, 167, 170, 194, 199, 205, 206, 217, 218, 222, 224, 232, 235, 236, 245, 248, 252 and/or 274 (according to BPN′ numbering) and has proteolytic activity. In one embodiment, said protease comprises one or more further substitutions: (a) threonine at position 3 (3T), (b) isoleucine at position 4 (41), (c) alanine, threonine or arginine at position 63 (63A, 63T, or 63R), (d) aspartic acid or glutamic acid at position 156 (156D or 156E), (e) proline at position 194 (194P), (f) methionine at position 199 (199M), (g) iso-leucine at position 205 (2051), (h) aspartic acid, glutamic acid or glycine at position 217 (217D, 217E or 217G), (i) combinations of two or more amino acids according to (a) to (h). A suitable protease may be at least 80% identical to SEQ ID NO: 10 and is characterized by comprising one amino acid (according to (a)-(h)) or combinations according to (i) together with the amino acid 101E, 101D, 101N, 101Q, 101A, 101G, or 101S (according to BPN′ numbering) and has proteolytic activity. In one embodiment, the protease is at least 80% identical to SEQ ID NO:10 and is characterized by comprising the mutation (according to BPN′ numbering) R101E, or S3T+V4I+V205I, or S3T+V4I+R101E+V205I or S3T+V4I+V199M+V205I+L217D, and has proteolytic activity. In another embodiment, the protease comprises an amino acid sequence having at least 80% identity to SEQ ID NO: 10 and being further characterized by comprising S3T+V4I+S9R+A15T+V68A+D99S+R101S+A103S+I104V+N218D (according to the BPN′ numbering) and has proteolytic activity. In another embodiment, the protease may have an amino acid sequence being at least 80% identical to SEQ ID NO:10 and being further characterized by comprising R101E, and one or more substitutions selected from the group consisting of S156D, L262E, Q137H, S3T, R45E,D,Q, P55N, T58W,Y,L, Q59D,M,N,T, G61 D,R, S87E, G97S, A98D,E,R, S106A,W, N117E, H120V,D,K,N, S125M, P129D, E136Q, S144W, S161T, S163A,G, Y171 L, A172S, N185Q, V199M, Y209W, M222Q, N238H, V244T, N261T, D and L262N, Q,D (according to the BPN′ numbering), and has proteolytic activity.

Further examples of useful proteases are the variants described in: WO 92/19729, WO 96/034946, WO 98/20115, WO 98/20116, WO 99/11768, WO 01/44452, WO 03/006602, WO 2004/03186, WO 2004/041979, WO 2007/006305, WO 2011/036263, WO 2011/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V41, S9R, A15T, K27R, *36D, V68A, N76D, N87S,R, *97E, A98S, S99G,D,A, S99AD, S101G,M,R S103A, V104I.Y.N, S106A, G118V.R, H120D.N, N123S, S128L, P129Q, S130A, G160D, Y167A, R170S, A194P, G195E, V199M, V205I, L217D, N218D, M222S, A232V, K235L, Q236H, Q245R, N252K, T274A (using BPN′ numbering).

Suitable commercially available proteases include those sold under the trade names Alcalase®, Blaze®; Duralase™, Durazyrn™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® all could be sold as Ultra® or Evity® (Novozymes A/S), those sold under the tradename Maxatase®, Maxacai®, Maxapem®, Purafect®, Purafect Prime®, Preferenz™, Purafect MA®, Purafect Ox®, Purafect OxP®, Puramax®, Properase®, Effectenz™, FN2®, FN3®, FN4®, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Lipases

“Lipases”, “lipolytic enzyme”, “lipid esterase”, all refer to an enzyme of EC class 3.1.1 (“carboxylic ester hydrolase”). Lipase means active protein having lipase activity (or lipolytic activity; triacylglycerol lipase, EC 3.1.1.3), cutinase activity (EC 3.1.1.74; enzymes having cutinase activity may be called cutinase herein), sterol esterase activity (EC 3.1.1.13) and/or wax-ester hydrolase activity (EC 3.1.1.50). Lipases include those of bacterial or fungal origin.

In one aspect of the invention, a suitable lipase is selected from the following: lipases from Humicola (synonym Thermomyces), e.g. from H. lanuginosa (T. lanuginosus) as described in EP 258068, EP 305216, WO 92/05249 and WO 2009/109500 or from H. insolens as described in WO 96/13580; lipases derived from Rhizomucor miehei as described in WO 92/05249; lipase from strains of Pseudomonas (some of these now renamed to Burkholderia), e.g. from P. alcaligenes or P. pseudoalcaligenes (EP 218272, WO 94/25578, WO 95/30744, WO 95/35381, WO 96/00292), P. cepacia (EP 331376), P. stutzeri (GB 1372034), P. fluorescens, Pseudomonas sp. strain SD705 (WO 95/06720 and WO 96/27002), P. wisconsinensis (WO 96/12012), Pseudomonas mendocina (WO 95/14783), P. glumae (WO 95/35381, WO 96/00292); lipase from Streptomyces griseus (WO 2011/150157) and S. pristinaespiralis (WO 2012/137147), GDSL-type Streptomyces lipases (WO 2010/065455); lipase from Thermobifida fusca as disclosed in WO 2011/084412; lipase from Geobacillus stearothermophilus as disclosed in WO 2011/084417; Bacillus lipases, e.g. as disclosed in WO 00/60063, lipases from B. subtilis as disclosed in Dartois et al. (1992), Biochemica et Biophysica Acta, 1131, 253-360 or WO 2011/084599, B. stearothermophilus (JP S64-074992) or B. pumilus (WO 91/16422); lipase from Candida antarctica as disclosed in WO 94/01541; cutinase from Pseudomonas mendocina (U.S. Pat. No. 5,389,536, WO 88/09367); cutinase from Magnaporthe grisea (WO 2010/107560); cutinase from Fusarum solani pisi as disclosed in WO 90/09446, WO 00/34450 and WO 01/92502; and cutinase from Humicola lanuginosa as disclosed in WO 00/34450 and WO 01/92502.

Such suitable lipase variants are e.g. those which are developed by methods as disclosed in WO 95/22615, WO 97/04079, WO 97/07202, WO 00/60063, WO 2007/087508, EP 407225 and EP 260105.

Commercially available lipase enzymes include but are not limited to those sold under the trade names Lipolase™, Lipex™, Lipolex™ and Lipoclean™ (Novozymes A/S), Lumafast (originally from Genencor), Preferenz L (DuPont), and Lipomax (Gist-Brocades/now DSM).

In one embodiment, lipase is selected from fungal triacylglycerol lipase (EC class 3.1.1.3). Fungal triacylglycerol lipase may be selected from lipases of Thermomyces lanuginosus. In one embodiment, the Thermomyces lanuginosa lipase is selected from triacylglycerol lipase according to amino acids 1-269 of SEQ ID NO: 2 of U.S. Pat. No. 5,869,438 and variants thereof having lipolytic activity.

Thermomyces lanuginosa lipase may be selected from variants having lipolytic activity which are at least 90%, at least 91%, at least 92%, at least 93%, at least 94%, at least 95%, at least 96%, at least 97%, at least 98%, at least 99%, or 100% identical when compared to the full-length polypeptide sequence of amino acids 1-269 of SEQ ID NO: 2 of U.S. Pat. No. 5,869,438.

Thermomyces lanuginosa lipase may be selected from variants having lipolytic activity comprising conservative mutations only, which do not pertain the functional domain of amino acids 1-269 of SEQ ID NO: 2 of U.S. Pat. No. 5,869,438. Lipase variants of this embodiment having lipolytic activity may be at least 95%, at least 96%, at least 97%, at least 98%, or at least 99% similar when compared to the full-length polypeptide sequence of amino acids 1-269 of SEQ ID NO: 2 of U.S. Pat. No. 5,869,438.

Thermomyces lanuginosa lipase may be selected from variants having lipolytic activity comprising the following amino acid substitutions when compared to amino acids 1-269 of SEQ ID NO: 2 of U.S. Pat. No. 5,869,438: T231R and N233R. Said lipase variants may further comprise one or more of the following amino acid exchanges when compared to amino acids 1-269 of SEQ ID NO: 2 of U.S. Pat. No. 5,869,438: Q4V, V60S, A150G, L227G, P256K.

Thermomyces lanuginosa lipase may be selected from variants having lipolytic activity comprising the amino acid substitutions T231R, N233R, Q4V, V60S, A150G, L227G, P256K within the polypeptide sequence of amino acids 1-269 of SEQ ID NO: 2 of U.S. Pat. No. 5,869,438 and are at least 95%, at least 96%, or at least 97% similar when compared to the full-length polypeptide sequence of amino acids 1-269 of SEQ ID NO: 2 of U.S. Pat. No. 5,869,438.

Thermomyces lanuginosa lipase may be selected from variants having lipolytic activity comprising the amino acid substitutions T231R and N233R within amino acids 1-269 of SEQ ID NO: 2 of U.S. Pat. No. 5,869,438 and are at least 95%, at least 96%, at least 97%, at least 98%, or at least 99% similar when compared to the full-length polypeptide sequence of amino acids 1-269 of SEQ ID NO: 2 of U.S. Pat. No. 5,869,438.

Thermomyces lanuginosa lipase may be a variant of amino acids 1-269 of SEQ ID NO: 2 of U.S. Pat. No. 5,869,438 having lipolytic activity, wherein the variant of amino acids 1-269 of SEQ ID NO: 2 of U.S. Pat. No. 5,869,438 is characterized in containing the amino acid substitutions T231R and N233R. Thermomyces lanuginosa lipase may be selected from variants having lipolytic activity preferably comprising at least one, preferably more than one, more preferably all of the following substitutions N11K, A18K, G23K, K24A, V771, D130A, V1541, V187T, T189Q within the polypeptide sequence of amino acids 1-269 of SEQ ID NO: 1 of WO2015/010009 and are at least 95%, at least 96%, or at least 97% similar when compared to the full length polypeptide sequence of amino acids 1-269 of SEQ ID NO: 1 of WO2015/010009.

### Amylases

Amylases include those of bacterial or fungal origin (EC 3.2.1.1 and 3.2.1.2, respectively). Preferably, amylases are selected from the group of alpha-amylases (EC 3.2.1.1).

Amylases maybe from Bacillus licheniformis having SEQ ID NO:2 as described in WO 95/10603 and variants at least 95% thereto. Suitable variants are described in WO 95/10603 comprising one or more substitutions in the following positions: 15, 23, 105, 106, 124, 128, 133, 154, 156, 178, 179, 181, 188, 190, 197, 201, 202, 207, 208, 209, 211, 243, 264, 304, 305, 391, 408, and 444 which have amylolytic activity. Variants are described in WO 94/02597, WO 94/018314, WO 97/043424 and SEQ ID NO:4 of WO 99/019467.

Amylases further maybe from B. stearothermophilus having SEQ ID NO:6 as disclosed in WO 02/10355 or an amylase with optionally having a C-terminal truncation over the wildtype sequence. Suitable variants of SEQ ID NO:6 include those comprising a deletion in positions 179 and/or 181 and/or 182 and/or a substitution in position 193.

Amylases further maybe from Bacillus sp.707 having SEQ ID NO:6 as disclosed in WO 99/19467 and variants at least 95% thereto. Preferred variants of SEQ NO: 6 are those having a substitution, a deletion or an insertion in one or more of the following positions: R181, G182, H183, G184, N195, 1206, E212, E216 and K269.

Amylases further maybe from Bacillus halmapalus having SEQ ID NO:2 or SEQ ID NO:7 as described in WO 96/23872, also described herein as SP-722. Preferred variants are described in WO 97/3296, WO 99/194671 and WO 2013/001078.

Amylases further may be from Bacillus sp. DSM 12649 having SEQ ID NO:4 as disclosed in WO 00/22103 and variants at least 95% thereto.

Amylases further may be from Bacillus sp. A 7-7 (DSM 12368) having an amino acid sequence at least 95% identical to SEQ ID NO:2, in particular over the region of the amino acids 32 to 516 according to SEQ ID NO:2, as disclosed in WO 02/10356.

Amylases further may be from Bacillus strain TS-23 having SEQ ID NO:2 as disclosed in WO 2009/061380 and variants thereof.

Amylases further may be from Cytophaga sp. having SEQ ID NO:1 as disclosed in WO 2013/184577 and variants at least 95% thereto.

Amylases further may be from Bacillus megaterium DSM 90 having SEQ ID NO:1 as disclosed in WO 2010/104675 and variants at least 95% thereto.

Amylases further may be from Bacillus sp. comprising amino acids 1 to 485 of SEQ ID NO:2 as described in WO 00/60060 and variants at least 95% thereto.

Amylases further may be from Bacillus amyloliquefaciens or variants thereof, preferably selected from amylases according to SEQ ID NO: 3 as described in WO 2016/092009.

Amylases may have SEQ ID NO:12 as described in WO 2006/002643 or amylase variants thereof comprising the substitutions Y295F and M202LITV within said SEQ ID NO:12. Amylases may have SEQ ID NO:6 as described in WO 2011/098531 or amylase variants comprising a substitution at one or more positions selected from the group consisting of 193 [G,A,S,T or M], 195 [F,W,Y,L,I or V], 197 [F,W,Y,L,I or V], 198 [Q or N], 200 [F,W,Y,L,I or V], 203 [F,W,Y,L,I or V], 206 [F,W,Y,N,L,I,V,H,Q,D or E], 210 [F,W,Y,L,I or V], 212 [F,W,Y,L,I or V], 213 [G,A,S,T or M] and 243 [F,W,Y,L,I or V] within said SEQ ID NO:6.

Amylases may have SEQ ID NO:1 as described in WO 2013/001078 or amylase variants comprising an alteration at two or more (several) positions corresponding to positions G304, W140, W189, D134, E260, F262, W284, W347, W439, W469, G476, and G477 within said SEQ ID NO: 1.

Amylases may have SEQ ID NO:2 as described in WO 2013/001087 or amylase variants comprising a deletion of positions 181+182, or 182+183, or 183+184, within said SEQ ID NO:2, optionally comprising one or two or more modifications in any of positions corresponding to W140, W159, W167, Q169, W189, E194, N260, F262, W284, F289, G304, G305, R320, W347, W439, W469, G476 and G477 within said SEQ ID NO:2.

Amylases may be hybrid alpha-amylases from above mentioned amylases as for example as described in WO 2006/066594.

Hybrid amylases may be according to WO 2014/183920 with A and B domains having at least 90% identity to SEQ ID NO:2 of WO 2014/183920 and a C domain having at least 90% identity to SEQ ID NO:6 of WO 2014/183920, wherein the hybrid amylase has amylolytic activity; preferably the hybrid alpha-amylase is at least 95% identical to SEQ ID NO: 23 of WO 2014/183920 and having amylolytic activity.

Hybrid amylases may be according to WO 2014/183921 with A and B domains having at least 75% identity to SEQ ID NO: 2, SEQ ID NO: 15, SEQ ID NO: 20, SEQ ID NO: 23, SEQ ID NO: 29, SEQ ID NO: 26, SEQ ID NO: 32, and SEQ ID NO: 39 as disclosed in WO 2014/183921 and a C domain having at least 90% identity to SEQ ID NO: 6 of WO 2014/183921, wherein the hybrid amylase has amylolytic activity; preferably, the hybrid alpha-amylase is at least 95% identical to SEQ ID NO: 30 as disclosed in WO 2014/183921 and having amylolytic activity;

Hybrid amylases may be according to WO 2021/032881 comprising an A and B domain originating from the alpha amylase originating from Bacillus sp. A 7-7 (DSM 12368) and a C domain originating from the alpha-amylase from Bacillus cereus; preferably, the A and B domain are at least 75% identical to the amino acid sequence of SEQ ID NO: 42 and a C domain is at least 75% identical to the amino acid sequence of SEQ ID NO: 44-both sequences as disclosed in WO 2021/032881; more preferably, the hybrid amylase is at least 80% identical to SEQ ID NO: 54 as disclosed in WO 2021/032881.

In one embodiment, at least one amylase is selected from commercially available amylases which include but are not limited to products sold under the trade names Duramyl™, Termamyl™, Fungamyl™, Stainzyme™, Stainzyme Plus™, Natalase™, Liquozyme X and BAN™, Amplify™, Amplify Prime™ (from Novozymes A/S), and Rapidase™, Purastar™, Powerase™, Effectenz™ (M100 from DuPont), Preferenz™ (S1000, S110 and F1000; from DuPont), PrimaGreen™ (ALL; DuPont), Optisize™ (DuPont), ENZYSIZE®, OPTISIZE HT PLUS®, PURASTAR OXAM® (Danisco/DuPont) and KAM® (Kao).

### Mannanases

“Mannanase” as described herein are enzymes selected from the group of mannan degrading enzyme. The mannan degrading enzyme may be selected from β-mannosidase (EC 3.2.1.25), endo-1,4-B-mannosidase (EC 3.2.1.78), and 1,4-β-mannobiosidase (EC 3.2.1.100). Preferably, the mannan degrading enzyme is selected from the group of endo-1,4-B-mannosidase (EC 3.2.1.78), a group of enzymes which may be called endo-β-1,4-D-mannanase, β-mannanase, or mannanase herein.

The mannanase may be selected from alkaline mannanase of Family 5 or 26 (i.e., GH5 or GH26). The term “alkaline mannanase” is meant to encompass mannanases having an enzymatic activity of at least 40% of its maximum activity at a given pH ranging from 7 to 12, preferably 7.5 to 10.5.

The mannanase may be selected from mannanases originating from Bacillus organisms, such as described in JP-0304706 [beta-mannanase from Bacillus sp.], JP-63056289 [alkaline, thermostable beta-mannanase], JP-63036774 [Bacillus microorganism FERM P-8856 producing beta-mannanase and beta-mannosidase at an alkaline pH], JP-08051975 [alkaline beta-mannanases from alkalophilic Bacillus sp. AM-001], WO 97/11164 [mannanase from Bacillus amyloliquefaciens], WO 91/18974 [mannanase active at an extreme pH and temperature], WO 97/11164 [mannanase from Bacillus amyloliquefaciens], WO 2014/100018 [endo-(3-mannanase1 cloned from a Bacillus circulans or Bacillus lentus strain CMG1240 (Bleman1; see U.S. Pat. No. 5,476,775)]. Suitable mannanases are described in WO 99/064619].

The mannanase may be selected from mannanases originating from Trichoderma organisms, such as disclosed in WO 93/24622.

The mannanase may be selected from a commercially available mannanase such as Mannaway® (Novozymes A/S) or Preferenz® (M100) (DuPont).

### Cellulases

“Cellulases” are enzymes capable of hydrolysing of cellulose. Cellulases may be selected from cellobiohydrolase (1,4-P-D-glucan cellobiohydrolase, EC 3.2.1.91), endo-ss-1,4-glucanase (EC 3.2.1.4) and ss-glucosidase (EC 3.2.1.21). Endoglucanases of EC class 3.2.1.4 may be named endoglucanase, endo-1,4-ss-D-glucan 4-glucano hydrolase, endo-1,4-beta-glucanase, carboxymethyl cellulase, and beta-1,4-glucanase.

Endoglucanases may be classified by amino acid sequence similarities (Henrissat, B. Accessed at UniProt Oct. 26, 2011) under family 5 containing more than 20 endoglucanases of EC 3.2.1.4. Reference is also made to T.-M. Enveri, “Microbial Cellulases” in W. M. Fogarty, Microbial Enzymes and Biotechnology, Applied Science Publishers, p. 183-224 (1983); Methods in Enzymology, (1988) Vol. 160, p. 200-391 (edited by Wood, W. A. and Kellogg, S. T.); Béguin, P., “Molecular Biology of Cellulose Degradation”, Annu. Rev. Microbiol. (1990), Vol. 44, pp. 219248; Begun, P. and Aubert, J-P., “The biological degradation of cellulose”, FEMS Microbiology Reviews 13 (1994) p. 25-58; Henrissat, B., “Cellulases and their interaction with cellulose”, Cellulose (1994), Vol. 1, pp. 169-196.

Preferably, at least one cellulase comprised in the formulation of the invention is selected of the glycosyl hydrolase family 7 (GH7, pfam00840), preferably selected from endoglucanases (EC 3.2.1.4).

Preferably, an alkaline cellulase is used, wherein “alkaline cellulase” is meant to encompass cellulases having enzymatic activity at a given pH ranging from 7 to 12, preferably 7.5 to 10.5. In one embodiment, the cellulase is selected from cellulases comprising a cellulose binding domain. In one another embodiment, the cellulase comprises a catalytic domain, but no cellulose binding domain.

In one embodiment, the formulation of the invention comprises at least one endoglucanases of EC class 3.2.1.4 is originating from
Bacillus, such as Bacillus sp. CBS 670.93 and CBS 669.93
Melanocarpus, such as Melanocarpus albomyces as disclosed in WO 97/14804
Clostridium, e.g. Clostridium thermocellum
Humicola, such as Humicola insolens (DSM1800) as disclosed in EP 0495257, EP 0531315, EP 0531372, U.S. Pat. Nos. 4,435,307, 5,648,263, 5,776,757, WO 89/09259, WO 91/17244, WO 94/07998 (sequence displayed in FIG. 1 43kd human variants thereof), WO 95/24471, WO 96/11262 and WO 98/12307.
Fusarium, such as Fusarium oxysporum e.g. strain J79 (DSM2672) as disclosed in EP 0495257, EP 0531315, EP 0531372, U.S. Pat. Nos. 5,648,263, 5,776,757, WO 89/09259, WO 91/17244, WO 95/24471 and WO 96/11262.
Thielavia, such as Thielavia terrestris or Myceliophthora thermophila strain CBS 11765 as disclosed in EP 0531315, U.S. Pat. Nos. 5,648,263, 5,776,757, WO 89/09259, WO 91/17244, WO 95/24471, WO 96/11262, WO 96/29397 (SEQ ID NO: 9 and variants thereof), and WO 98/12307.
Trichoderma, such as Trichoderma reesei, Trichoderma longibrachiatum or Trichoderma harzianum as disclosed in EP 1305432, EP 1240525, WO 92/06165, WO 94/21801, WO 94/26880, WO 95/02043, WO 95/24471 and WO 02/099091.
Aspergillus, such as Aspergillus aculeatus as disclosed in WO 93/17244.
Erwinia, such as Erwinia chrysanthermi as described by M. H. Boyer et. al. in European Journal of Biochemistry, vol. 162, page 311-316 (1987).
Acremonium such as Acremonium sp., Acremonium persicinum, Acremonium acremonium, Acremonium brachypenium, Acremonium dichromosporum, Acremonium obclavatum, Acremonium pinkertoniae, Acremonium roseogriseum, Acremonium incoloratum, and Acremonium furatum as disclosed in WO 96/11262 and WO 96/29397 (SEQ ID NO: 5 and variants thereof).
Cellvibrio such as Cellvibrio mixtus DSM 11683, Cellvibrio mixtus DSM 11684, Cellvibrio mixtus DSM 11685, Cellvibrio mixtus ACM 2601, Cellvibrio mixtus DSM 1523, and Cellvib-rio gilvus DSM 11686, as disclosed in WO 98/08940.
Cephalosporium, such as Cephalosporium sp. RYM-202 as disclosed in WO 96/11262.

Suitable cellulases include also those, which are variants of the above described cellulases which have cellulolytic activity. In one embodiment cellulase variants include variants with at least 40 to 100% identity when compared to the full-length polypeptide sequence of the parent enzyme as disclosed above. In one embodiment cellulase variants having cellulolytic activity are at least 70%, at least 75%, at least 80%, at least 85%, at least 90%, at least 91%, at least 92%, at least 93%, at least 94%, at least 95%, at least 96%, at least 97%, at least 98% or at least 99% similar and/or identical to the full-length polypeptide sequence of the parent enzyme as disclosed above.

The cellulase may be a Humicola insolens DSM 1800 cellulase complex having endoglucanase, cellobiohydrolase and beta-glucosidase activity.

The cellulase may be a Humicola insolens DSM 1800 endoglucanase (EC 3.2.1.4), preferably having the polypeptide sequence according to position 21-435 of SEQ ID NO:2 as disclosed in WO 2018/224544 or variants at least 95% identical thereto.

The cellulase may be a Humicola insolens endoglucanase (EC 3.2.1.4) having 43 kD, preferably according to the polypeptide sequence as disclosed in FIG. 1a of WO 94/07998 (“43kDhum”) or variants thereof which are preferably at least 90% identical thereto, preferably those disclosed in WO 94/07998.

The cellulase may be a Bacillus sp. cellulase (EC 3.2.1.4) selected from a polypeptide at least 80% similar and/or identical to the amino acid sequence of position 1 to position 773 of SEQ ID NO: 2 of WO 2004/053039 or a catalytically active fragment thereof. In one embodiment, the cellulase is a mature polypeptide which is at least 95% identical to SEQ ID NO:1 of WO 2018/224544.

The cellulase may be a Thielavia terrestris cellulase (EC 3.2.1.4) having a polypeptide at least 80% similar and/or identical to the amino acid sequence of position 1 to position 299 of SEQ ID NO: 4 of WO 2004/053039 or a catalytically active fragment thereof. In one embodiment, the cellulase is a mature polypeptide which is at least 95% identical to SEQ ID NO:4 of WO 2018/224544.

The cellulase may be a mature Sordaria fimicola cellulase, preferably having a polypeptide sequence according to SEQ ID NO:5 of WO 2018/224544 or variants at least 95% identical thereto.

At least one cellulase may be selected from Renozyme®, Celluzyme®, Celluclean®, Endolase® and Carezyme® (Novozymes A/S), Clazinase™, and Puradax HA™ (Genencor Int. Inc.), and KAC-500 (B)™ (Kao Corporation).

### Enzyme Stabilizers

Care composition ingredients according to this invention include enzyme stabilizers. Enzyme stabilizers include water-soluble sources of calcium and/or magnesium ions like inorganic salts including CaCl₂), MgCl₂, or NaCl, polyols such 3-propanediol, ethylene glycol, glycerol, 1,2-propanediol, sugars, sugar alcohols like sorbitol, peptide aldehydes, short chain carboxylic acids and salts thereof like calcium formate or sodium formate, lactic acid, boric acid, boric acid derivatives like aromatic borate esters, phenyl boronic acid derivatives such as 4-formyl phenyl boronic acid, phenylboronic acid and derivatives thereof.

Enzyme stabilizers further include, peptide acetals, and peptide aldehyde hydrosulfite adducts and peptide aldehydes, for example of the formula B₂-B₁-B₀-R wherein R is hydrogen, CH₃, CX₃, CHX₂, or CH₂X, wherein X is a halogen atom, Bₒ is a phenylalanine residue with an OH substituent at the p-position and/or at the m-position, B₁ is a single amino acid residue and B₂ consists of one or more amino acid residues, optionally comprising an N-terminal protection group. Preferred peptide aldehydes include but are not limited to: Z-RAY-H, Ac-GAY-H, Z-GAY-H, Z-GALH, Z-GAF-H, Z-VAL-H, Z-GAV-H, Z-RVY-H, Z-LVY-H, Ac-LGAY-H, Ac-FGAY-H, Ac-YGAY-H, Ac-FGVY-H or Ac-WLVY-H, where Z is benzyloxycarbonyl and Ac is acetyl.

### Dye Transfer Inhibiting Agents

Care composition ingredients according to this invention include dye transfer inhibiting agents. Dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof.

### Fabric Softeners

Care composition ingredients according to this invention include fabric softeners. Fabric softeners include smectite clays like montmorillonite clay, polydimethylsiloxane (PDMS), distearyldimethylammonium chloride (DSDMAC), diethyl ester dimethyl ammonium chloride (DEEDMAC), TEAQ (triethanolamine quat), HEQ (Hamburg esterquat), distearyldimethylammonium chloride (DSDMAC), salts of monoesters and diesters of phosphoric acid and fatty alcohols, ethoxylated phosphate esters.

### Hydrotopes

Care composition ingredients according to this invention include hydrotropes. Hydrotopes include anionic hydrotropes like sodium, potassium, and ammonium xylene sulfonate, sodium, potassium and ammonium toluene sulfonate, sodium, potassium, and ammonium cumene sulfonate, mixtures thereof, and related compounds, as disclosed in U.S. Pat. No. 3,915,903.

### Light Stabilizers

Care composition ingredients according to this invention include light stabilizers like Didodecyl 3,3′-thiodipropionate (e.g. Tinogard® Q).

### Malodor Control Agents

Care composition ingredients according to this invention include malodor control agents. Malodor control agents include ricinoleic acid, ricinoleic salts, ricinoleic esters, thymol, quaternary ammonium salts such as Bardac®, polyethylenimines (such as Lupasol®) and zinc complexes thereof, silver and silver compounds, especially those designed to slowly release Ag+ or nanosilver dispersions.

### Perfumes and Fragrances

Care composition ingredients according to this invention include fragrances, perfumes, and perfume raw materials. Perfume raw materials include 1,1′-oxybis-2-propanol; 1,4-cyclohexanedicarboxylic acid, diethyl ester; (ethoxymethoxy)cyclododecane; 1,3-nonanediol, monoacetate; (3-methylbutoxy) acetic acid, 2-propenyl ester; beta-methyl cyclododecaneethanol; 2-methyl-3-[(1,7,7-trimethylbicyclo[2.2.1] hept-2-yl)oxy]-1-propanol; oxacyclohexadecan-2-one; alpha-methyl-benzenemethanol acetate; trans-3-ethoxy-1,1,5-trimethylcyclohexane; 4-(1,1-dimethylethyl)cyclohexanol acetate; dodecahydro-3a,6,6,9a-tetramethylnaphtho[2,1-b]furan; beta-methyl benzenepropanal; beta-methyl-3-(1-methylethyl)benzenepropanal; 4-phenyl-2-butanone; 2-methylbutanoic acid, ethyl ester; benzaldehyde; 2-methylbutanoic acid, 1-methylethyl ester; dihydro-5-pentyl-2 (3H) furanone; (2E)-1-(2,6,6-trimethyl-2-cyclohexen-1-yl)-2-buten-1-one; dodecanal; undecanal; 2-ethyl-alpha, alpha-dimethylbenzenepropanal; decanal; alpha, alpha-dimethylbenzeneethanol acetate; 2-(phenylmethylene) octanal; 2-[[3-[4-(1,1-dimethylethyl)phenyl]-2-methylpropylidene]amino]benzoic acid, methyl ester; 1-(2,6,6-trimethyl3-cyclohexen-1-yl)-2-buten-1-one; 2-pentylcyclopentanone; 3-oxo-2-pentyl cyclopentaneacetic acid, methyl ester; 4-hydroxy-3-methoxybenzaldehyde; 3-ethoxy-4-hydroxybenzaldehyde; 2-heptylcyclopentanone; 1-(4-methylphenyl) ethanone; (3E)-4-(2,6,6-trimethyl-1-cyclohexen-1-yl)3-buten-2-one; (3E)-4-(2,6,6-trimethyl-2-cyclohexen-1-yl)-3-buten-2-one; benzeneethanol; 2H-1-benzopyran-2-one; 4-methoxybenzaldehyde; 10-undecenal; propanoic acid, phenylmethyl ester; beta-methylbenzenepentanol; 1,1-diethoxy-3,7-dimethyl-2,6-octadiene; alpha, alpha-dimethylbenzeneethanol; (2E)-1-(2,6,6-trimethyl-1-cyclohexen-1-yl)-2-buten-1-one; acetic acid, phenylmethyl ester; cyclohexanepropanoic acid, 2-propenyl ester; hexanoic acid, 2-propenyl ester; 1,2-dimethoxy-4-(2-propenyl)benzene; 1,5-dimethyl-bicyclo[3.2.1] octan-8-one oxime; 4-(4-hydroxy-4-methylpentyl)-3-cyclohexene-1-carboxaldehyde; 3-buten-2-ol; 2-[[[2,4 (or 3,5)-dimethyl-3-cyclohexen-1-yl]methylene]amino]benzoic acid, methyl ester; 8-cyclohexadecen-1-one; methyl ionone; 2,6-dimethyl-7-octen-2-ol; 2-methoxy-4-(2-propenyl) phenol; (2E)-3,7-dimethyl2,6-Octadien-1-ol; 2-hydroxy-Benzoic acid, (3Z)-3-hexenyl ester; 2-tridecenenitrile; 4-(2,2-dimethyl-6-methylenecyclohexyl)-3-methyl-3-buten-2-one; tetrahydro-4-methyl-2-(2-methyl-1-propenyl)-2H-pyran; Acetic acid, (2-methylbutoxy)-, 2-propenyl ester; Benzoic acid, 2-hydroxy, 3-methylbutyl ester; 2-Buten-1-one, 1-(2,6,6-trimethyl-1-cyclohexen-1-yl)-, (Z)—; Cyclopentanecarboxylic acid, 2-hexyl-3-oxo-, methyl ester; Benzenepropanal, 4-ethyl-alpha, alpha-dimethyl-; 3-Cyclohexene-1-carboxaldehyde, 3-(4-hydroxy-4-methylpentyl)-; Ethanone, 1-(2,3,4,7,8,8a hexahydro-3,6,8,8-tetramethyl-1H-3a,7-methanoazulen-5-yl)-, [3R-(3. alpha., 3a. beta., 7. beta., 8a. alpha.)]-; Undecanal, 2-methyl-2H-Pyran-2-one, 6-butyltetrahydro-; Benzenepropanal, 4-(1,1-dimethylethyl)-. alpha.-methyl-; 2 (3H)-Furanone, 5-heptyldihydro-; Benzoic acid, 2-[(7-hydroxy-3,7-dimethyloctylidene)amino]-, methyl; Benzoic acid, 2-hydroxy-, phenylmethyl ester; Naphthalene, 2-methoxy-; 2-Cyclopenten-1-one, 2-hexyl-; 2 (3H)-Furanone, 5-hexyldihydro-; Oxiranecarboxylic acid, 3-methyl-3-phenyl-, ethyl ester; 2-Oxabicyclo[2.2.2] octane, 1,3,3-trimethyl-; Benzenepentanol, gamma-methyl-; 3-Octanol, 3,7-dimethyl-; 3,7-dimethyl-2,6-octadienenitrile; 3,7-dimethyl-6-octen-1-ol; Terpineol acetate; 2-methyl-6-methylene-7-Octen-2-ol, dihydro derivative; 3a,4,5,6,7,7a-hexahydro-4,7-Methano-1H-inden-6-ol propanoate; 3-methyl-2-buten-1-ol acetate; (Z)-3-Hexen-1-ol acetate; 2-ethyl-4-(2,2,3-trimethyl-3-cyclopenten1-yl)-2-buten-1-ol; 4-(octahydro-4,7-methano-5H-inden-5-ylidene)-butanal; 3-2,4-dimethylcyclohexene-1-carboxaldehyde; 1-(1,2,3,4,5,6,7,8-octahydro-2,3,8,8-tetramethyl-2-naphthalenyl)-ethanone; 2-hydroxy-benzoic acid, methyl ester; 2-hydroxy-benzoic acid, hexyl ester; 2-phenoxy-ethanol; 2-hydroxy-benzoic acid, pentyl ester; 2,3-heptanedione; 2-hexen-1-ol; 6-Octen-2-ol, 2,6-dimethyl-; damascene (alpha, beta, gamma or delta or mixtures thereof), 4,7-Methano-1H-inden-6-ol, 3a,4,5,6,7,7a-hexahydro-, acetate; 9-Undecenal; 8-Undecenal; Isocyclocitral; Ethanone, 1-(1,2,3,5,6,7,8,8a-octahydro-2,3,8,8-tetramethyl-2-naphthalenyl)-; 3-Cyclohexene-1-carboxaldehyde, 3,5-dimethyl-; 3-Cyclohexene-1-carboxaldehyde, 2,4-dimethyl-; 1,6-Octadien-3-ol, 3,7-dimethyl-; 1,6-Octadien-3-ol, 3,7-dimethyl acetate; Lilial (p-t-Bucinal), and Cyclopentanone, 2-[2-(4-methyl-3-cyclohexen-1-yl) propyl]-and 1-methyl-4-(1-methylethenyl)cyclohexene and mixtures thereof.

### Photocatalytic Systems

Care composition ingredients according to this invention include photocatalytic systems. Photocatalytic systems comprise Tetrabenzo tetraazoporphyrines like the ones commercially available under the trade names Tinolux® BBS, Tinolux® BMC Liquid, and Tinolux® BBS Solid.

### Pigments

Care composition ingredients according to this invention include pigments. Pigments include compounds selected from the group consisting of flavanthrone, indanthrone, chlorinated indanthrone containing from 1 to 4 chlorine atoms, pyranthrone, dichloropyranthrone, monobromodichloropyranthrone, dibromodichloropyranthrone, tetrabromopyranthrone, perylene-3,4,9,10-tetracarboxylic acid diimide, wherein the imide groups may be unsubstituted or substituted by C₁-C₃-alkyl or a phenyl or heterocyclic radical, and wherein the phenyl and heterocyclic radicals may additionally carry substituents which do not confer solubility in water, anthrapyrimidinecarboxylic acid amides, violanthrone, isoviolanthrone, dioxazine pigments, copper phthalocyanine which may contain up to 2 chlorine atoms per molecule, polychloro-copper phthalocyanine or polybromochloro-copper phthalocyanine containing up to 14 bromine atoms per molecule and mixtures thereof. Pigments further include pigments selected from the group consisting of Ultramarine Blue (C.I. Pigment Blue 29), Ultramarine Violet (C.I. Pigment Violet 15) and mixtures thereof.

### Polymeric Grease Cleaning Agents

Care composition ingredients according to this invention include amphiphilic alkoxylated grease cleaning polymers which have balanced hydrophilic and hydrophobic properties such that they remove grease particles from fabrics and surfaces. Polymeric grease cleaning agents include alkoxylated polyalkylenimines, preferably having an inner polyethylene oxide block and an outer polypropylene oxide block. Polymeric grease cleaning agents include alkoxylated polycarboxylates These materials comprise polyacrylates having one alkoxy sidechain per every 7-8 acrylate units. The side-chains are of the formula —(CH₂CH₂O)ₘ(CH₂)ₙCH₃ wherein m is 2-3 and n is 6-12. The sidechains are ester-linked to the polyacrylate “backbone” to provide a “comb” polymer type structure. The molecular weight can vary but is typically in the range of 2,000 to 50,000.

### Solvents

Care composition ingredients according to this invention include solvents. Solvents include water and other solvents such as lipophilic fluids. Suitable lipophilic fluids include siloxanes, other silicones, hydrocarbons, glycol ethers, glycerin derivatives such as glycerin ethers, perfluorinated amines, perfluorinated and hydrofluoroether solvents, low-volatility nonfluorinated organic solvents, diol solvents, other environment-friendly solvents and mixtures thereof.

### Peroxide Sources

Care composition ingredients according to this invention include sources of hydrogen peroxide. Sources of hydrogen peroxide include inorganic perhydrate salts, including alkali metal salts such as sodium salts of perborate (usually mono- or tetra-hydrate), percarbonate, persulphate, perphosphate, persilicate salts and mixtures thereof.

### Rheology Modifiers

Care composition ingredients according to this invention include rheology modifiers. Rheology modifiers include, without being limited to, copolymers of methacrylic acid and acrylic acid esters (e.g. Rheovis® AT 120, Rheovis® TTA), cationic acrylic polymers (e.g. Rheovis® CDE Pro), cationically modifioed polyacrylamides (e.g. Rheovis® CSP), and cationically modifioed polyacrylates (e.g. Rheovis® FRC).

### Suds Suppressors

Care composition ingredients according to this invention include suds suppressors. Suds supressors include monocarboxylic fatty acid and soluble salts therein, high molecular weight hydrocarbons such as paraffin, fatty acid esters (e.g., fatty acid triglycerides), fatty acid esters of monovalent alcohols, aliphatic C₁₈-C₄₀ ketones (e.g stearone), N-alkylated amino triazines, waxy hydrocarbons preferably having a melting point below about 100° C., silicone suds suppressors, and secondary alcohols. Suds suppressors are described in U.S. Pat. Nos. 2,954,347; 4,265,779; 4,265,779; 3,455,839; 3,933,672; 4,652,392; 4,978,471; 4,983,316; 5,288,431; 4,639,489; 4,749,740; 4,798,679; 4,075,118; EP 89307851.9; EP 150872; and DOS 2,124,526.

### Surfactants

Care composition ingredients according to this invention include surfactants. Surfactants comprise anionic surfactants, non-ionic surfactants, amphoteric surfactants, zwitterionic surfactants, and cationic surfactants.

Anionic surfactants comprise sulfates and sulfonates, in particular C₉-C₂₀ linear alkyl benzene sulfonates (LAS), isomers of LAS, C₁₀-C₂₀ primary, branched chain and random alkyl sulfates (AS); C₁₀-C₁₈ secondary (2,3)alkyl sulfates; C₁₀-C₁₈ alkyl alkoxy sulfates (AEₓS) wherein x is from 1 to 30; C₁₀-C₁₈ alkyl alkoxy carboxylates comprising 1 to 5 ethoxy units, alkali metal and ammonium salts of C₈-C₁₂-alkyl sulfates, of C₁₂-C₁₈-fatty alcohol ether sulfates, of C₁₂-C₁₈-fatty alcohol polyether sulfates, of sulfuric acid half-esters of ethoxylated C₄-C₁₂-alkylphenols (ethoxylation: 3 to 50 mol of ethylene oxide/mol), of C₁₂-C₁₈-alkylsulfonic acids, of C₁₂-C₁₈ sulfo fatty acid alkyl esters, for example of C₁₂-C₁₈ sulfo fatty acid methyl esters, of C₁₀-C₁₈-alkylarylsulfonic acids, preferably of n-C₁₀-C₁₈-alkylbenzene sulfonic acids, of C₁₀-C₁₈ alkyl alkoxy carboxylates and of soaps such as for example C₈-C₂₄-carboxylic acids., branched alkyl benzene sulfonates (BABS), phenyl alkane sulfonates, alpha-olefin sulfonates (AOS), olefin sulfonates, alkene sulfonates, alkane-2,3-diylbis(sulfates), hydroxy alkane sulfonates and disulfonates, alkyl sulfates (AS) such as sodium dodecyl sulfate (SDS), fatty alcohol sulfates (FAS), primary alcohol sulfates (PAS), alcohol ether sulfates (AES or AEOS or FES, also known as alcohol ethoxy sulfates or fatty alcohol ether sulfates), secondary alkane sulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenyl succinic acid, dodecenyl/tetradecenyl succinic acid (DTSA), fatty acid derivatives of amino acids, diesters and monoesters of sulfo succinic acid.

Anionic surfactants further comprise the compounds in the table “Anionic surfactants” below.

### Table “Anionic Surfactants”

| Trade name | Chemical constitution | Chemical class |
|---|---|---|
| Texapon ® N 70 | C12C14-Fatty alcohol ethersulfate | Fatty Alcohol |
| | (2 EO), sodium salt | Ethersulfates |
| Texapon ® N | C12C14-Fatty alcohol ethersulfate | Fatty Alcohol |
| 701 | (1 EO), sodium salt | Ethersulfates |
| Texapon ® N | C12C14-Fatty alcohol ethersulfate | Fatty Alcohol |
| 703 | (3 EO), sodium salt | Ethersulfates |
| Texapon ® NSO | C12C14-Fatty alcohol ethersulfate | Fatty Alcohol |
| UP | (2 EO), sodium salt | Ethersulfates |
| Sulfopon ® 101 | C12C16-Fatty alcohol sulfate, | Fatty Alcohol |
| UP | sodium salt | Sulfates |
| Sulfopon ® 1214 | C12 C14-Fatty alcohol sulfate, | Fatty Alcohol |
| G | sodium salt | Sulfates |
| Sulfopon ® 1216 | C12C16-Fatty alcohol sulfate, | Fatty Alcohol |
| G | sodium salt | Sulfates |
| Sulfopon ® 1218 | C12C18-Fatty alcohol sulfate, | Fatty Alcohol |
| G | sodium salt | Sulfates |
| Sulfopon ® 8515 | C12C14-Fatty alcohol sulfate, | Fatty Alcohol |
| G | sodium salt | Sulfates |
| Texapon ® LS | C12C14-Fatty alcohol sulfate, | Fatty Alcohol |
| 30 | sodium salt | Sulfates |
| Texapon ® K 30 | C12C18-Fatty alcohol sulfate, | Fatty Alcohol |
| UP | sodium salt | Sulfates |
| Texapon ® EHS | 2-Ethylhexyl sulfate, sodium salt | Fatty Alcohol |
| | | Sulfates |
| Texapon ® 842 | C8-Fatty alcohol sulfate, sodium | Fatty Alcohol |
| UP | salt | Sulfates |
| Disponil ® | Linear C10C13-alkyl benzene | Linear Alkyl |
| LDBS 55 | sulfonate, sodium salt | Benzene |
| | | Sulfonates |
| Disponil ® SUS | | Di-isooctyl |
| IC 875 | | sulfosuccinate, |
| | | sodium salt |
| Maranil ® | Linear C10C13-alkyl benzene | Linear Alkyl |
| DBS/E | sulfonic acid | Benzene |
| | | Sulfonates |
| Maranil ® | Linear C10C13-alkyl benzene | Linear Alkyl |
| DBS/LC | sulfonic acid | Benzene |
| | | Sulfonates |
| Lutensit ® A-EP | | Acid phosphoric |
| | | ester of a fatty |
| | | alcohol ethoxylate |
| Lutensit ® A-BO | | fatty alcohol |
| | | ethoxylate |

Non-ionic surfactants comprise C₈-C₁₈ alkyl ethoxylates, such as, NEODOL®; C₆-C₁₂ alkyl phenol alkoxylates wherein the alkoxylate units may be ethyleneoxy units, propyleneoxy units or a mixture thereof; C₁₂-C₁₈ alcohol and C₆-C₁₂ alkyl phenol condensates with ethylene oxide/propylene oxide block polymers such as Pluronic®; C₁₄-C₂₂ mid-chain branched alcohols; C₁₄-C₂₂ mid-chain branched alkyl alkoxylates, typically having an average degree of alkoxylation of from 1 to 30; alkylpolysaccharides, alkylpolyglycosides, alkylmonoglycosides, polyhydroxy fatty acid amides; ether capped poly(oxyalkylated) alcohol surfactants, alkoxylated alcohols, in one aspect C₈-C₁₈ alkyl alkoxylated alcohol, e.g., a C₈-C₁₈ alkyl ethoxylated alcohol, the alkyl alkoxylated alcohol may have an average degree of alkoxylation of from 1 to 50, from 1 to 30, from 1 to 20, or from 1 to 10. In one aspect, the alkyl alkoxylated alcohol may be a C₈-C₁₈ alkyl ethoxylated alcohol having an average degree of ethoxylation of from 1 to 10, from 1 to 7, more from 1 to 5 or from 3 to 7. The alkyl alkoxylated alcohol can be linear or branched and substituted or unsubstituted. Nonionic surfactants include alcohol ethoxylates (AE or AEO), such as linear alcohol (C₁₂-C₁₅) ethoxylate (LAE), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN® and TWEEN®.

Non-ionic surfactants include C₈-C₁₈ alkyl ethoxylates, such as NEODOL® non-ionic surfactants from Shell; ethylenoxide/propylenoxide block alkoxylates as PLURONIC® from BASF; C₁₄-C₂₂ mid-chain branched alkyl alkoxylates, BAEx, wherein x is from 1 to 30, as disclosed in U.S. Pat. Nos. 6,153,577, 6,020,303 and 6,093,856; alkylpolysaccharides as disclosed in U.S. Pat. No. 4,565,647 Llenado, issued Jan. 26, 1986; specifically alkylpolyglycosides as disclosed in U.S. Pat. Nos. 4,483,780 and 4,483,779; polyhydroxy fatty acid amides as disclosed in U.S. Pat. No. 5,332,528; and ether capped poly(oxyalkylated) alcohol surfactants as disclosed in U.S. Pat. No. 6,482,994 and WO 01/42408.

Non-ionic surfactants include alkoxylated alcohols and alkoxylated fatty alcohols, di- and multiblock copolymers of ethylene oxide and propylene oxide and reaction products of sorbitan with ethylene oxide or propylene oxide, alkylphenol ethoxylates, alkyl glycosides, polyhydroxy fatty acid amides (glucamides)

Examples of alkoxylated alcohols and alkoxylated fatty alcohols are compounds of the general formula (A)

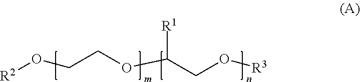

in which the variables are defined as follows:
R¹ is selected from linear C₁-C₁₀-alkyl, preferably ethyl and particularly preferably methyl,
R² is selected from C₈-C₂₂-alkyl, for example n-C₈H₁₇, n-C₁₀H₂₁, n-C₁₂H₂₅, n-C₁₄H₂₉, n-C₁₆H₃₃ or n-C₁₈H₃₇,

R³ is selected from C₁-C₁₀-alkyl, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl or isodecyl, m and n are in the range from zero to 300, where the sum of n and m is at least one. Preferably, m is in the range from 1 to 100 and n is in the range from 0 to 30.

Compounds of the general formula (A) may be block copolymers or random copolymers, preference being given to block copolymers.

Other examples of alkoxylated alcohols and alkoxylated fatty alcohols are, compounds of the general formula (B)

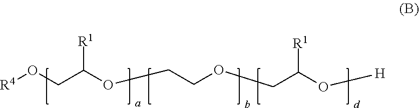

in which the variables are defined as follows:
R¹ is identical or different and selected from linear C₁-C₄-alkyl, preferably identical in each case and ethyl and particularly preferably methyl,
R⁴ is selected from C₆-C₂₀-alkyl, in particular n-C₈H₁₇, n-C₁₀H₂₁, n-C₁₂H₂₅, n-C₁₄H₂₉, n-C₁₆H₃, n-C₁₈H₃₇,
a is a number in the range from zero to 6, preferably 1 to 6,
b is a number in the range from zero to 20, preferably 4 to 20,
d is a number in the range from 4 to 25.

Preferably, at least one of a and b is greater than zero.

Compounds of the general formula (B) may be block copolymers or random copolymers, preference being given to block copolymers.

Further examples of non-ionic surfactants are di- and multiblock copolymers, composed of ethylene oxide and propylene oxide, ethoxylated or propoxylated sorbitan esters, alkylphenol ethoxylates, alkyl polyglycosides, polyhydroxy fatty acid amides (glucamides).

Nonionic surfactants comprise the compounds in the table “Nonionic surfactants” below.

### Table “Nonionic Surfactants”

| Trade name | Function | Chemical constitution |
|---|---|---|
| Degressal ® SD 20 | Foam Suppressors | Fatty alcohol alkoxylate |
| Degressal ® SD 40 | Foam Suppressors | Phosphoric ester |
| Dehydol ® 04 Deo | Emulsifier and | C8-Fatty alcohol + approx. 4 EO |
| | Solubilizers | |
| Dehydol ® 100 | Nonionic Surfactant | C12C18 Fatty alcohol + 9 EO |
| Dehydol ® 980 | Nonionic Surfactant | Fatty alkohol alkoxylate |
| Dehydol ® LS 6 | Nonionic Surfactant | C12C14 Fatty alcohol + 6 EO |
| Dehydol ® LT 6 | Nonionic Surfactant | C12C18 Fatty alcohol + 6 EO |
| Dehydol ® LT 7 | Nonionic Surfactant | C12C18 Fatty alcohol + 7 EO |

Dehypon ® type products below are fatty alcohol alkoxylates. They are alkoxylated which
provide very low foaming properties. The physical form vary from liquid to solid, even
granules are available. Dehypon ® grades are versatile useable and provide properties
such as wetting, soil removal, low foaming and emulsification.

| | | |
|---|---|---|
| Dehypon ® 2574 | Low-foaming Nonionic | Fatty alcohol alkoxylate |
| | Surfactants | |
| Dehypon ® E 126 | Low-foaming Nonionic | Fatty alcohol polyglycol ether, modified |
| | Surfactants | |
| Dehypon ® E 127 | Low-foaming Nonionic | Fatty alcohol polyglycol ether, modified |
| | Surfactants | |
| Dehypon ® G 2084 | Low-foaming Nonionic | Guerbet alcohol alkoxylate, end-capped |
| | Surfactants | |
| Dehypon ® GRA | Low-foaming Nonionic | Fatty alcohol polyglycol ether, modified |
| | Surfactants | |
| Dehypon ® KE 3447 | Low-foaming Nonionic | Fatty alcohol polyglycol ether, modified |
| | Surfactants | |
| Dehypon ® LS 24 | Low-foaming Nonionic | Fatty alcohol alkoxylate |
| | Surfactants | |
| Dehypon ® LS 36 | Low-foaming Nonionic | Fatty alcohol alkoxylate |
| | Surfactants | |
| Dehypon ® LS 54 | Low-foaming Nonionic | Fatty alcohol alkoxylate |
| | Surfactants | |
| Dehypon ® LT 104 | Low-foaming Nonionic | Fatty alcohol alkoxylate, end-capped |
| | Surfactants | |
| Dehypon ® OCP 502 | Foam Suppressors | Fatty alcohol alkoxylate |
| Dehypon ® Shine | Low-foaming Nonionic | |
| | Surfactants | |
| Dehypon ® WET | Low-foaming Nonionic | Fatty alcohol polyglycol ether, modified |
| | Surfactants | |
| Demelan ® GC 17 | Emulsifier and | Ethoxylated mono-/di glyceride |
| | Solubilizers | |
| Demelan ® VPC | Nonionic Surfactant | Blend of ethoxylated fatty amines and |
| | | ethoxylated fatty alcohols |
| Emulan ® A | Emulsifier and | Oleic acid ethoxylate |
| | Solubilizers | |
| Emulan ® AT 9 | Emulsifier and | Fatty alcohol ethoxylate |
| | Solubilizers | |
| Emulan ® EL | Emulsifier and | Castor oil ethoxylate |
| | Solubilizers | |
| Emulan ® TO 4070 | Emulsifier and | Fatty alcohol ethoxylate |
| | Solubilizers | |
| Glucopon ® 215 UP | Nonionic Surfactant | C8C10-Alkyl polyglucoside |
| Glucopon ® 225 DK | Nonionic Surfactant | C8C10-Alkyl polyglucoside |
| Glucopon ® 425 N/HH | Nonionic Surfactant | C8C16 -Alkyl polyglucoside |
| Glucopon ® 50 G | Nonionic Surfactant | C12C14 -Alkyl polyglucoside on |
| | | inorganic and organic carrier |
| Glucopon ® 600 CSUP | Nonionic Surfactant | C10C16-Alkyl polyglucoside |
| Glucopon ® 650 EC | Nonionic Surfactant | C8C16-Alkyl polyglucoside |
| Glucopon ® GD 70 | Nonionic Surfactant | C10C12-Alkyl polyglucoside |
| Lutensol ® A 7 N | Nonionic Surfactant | C12C14 Fatty alcohol + 7 EO |
| Lutensol ® AO 3 | Nonionic Surfactant | C13C15-Oxo alcohol + 3 EO |
| Lutensol ® AO 5 | Nonionic Surfactant | C13C15-Oxo alcohol + 5 EO |
| Lutensol ® AO 7 | Nonionic Surfactant | C13C15-Oxo alcohol + 7 EO |
| Lutensol ® AO 8 | Nonionic Surfactant | C13C15-Oxo alcohol + 8 EO |
| Lutensol ® AO 11 | Nonionic Surfactant | C13C15-Oxo alcohol + 11 EO |
| Lutensol ® AT 25 types | Nonionic Surfactant | C16C18-Fatty alcohol + 25 EO |
| Lutensol ® AT 50 types | Nonionic Surfactant | C16C18-Fatty alcohol + 50 EO |
| Lutensol ® AT 80 types | Nonionic Surfactant | C16C18-Fatty alcohol + 80 EO |
| Lutensol ® CS 6250 | Emulsifier and | Alcohol ethoxylate |
| | Solubilizers | |
| Lutensol ® FA 12 K | Nonionic Surfactant | Coco amine + approx 12 EO |
| Lutensol ® M 5 | Nonionic Surfactant | C10C18-Alcohol + 5 EO |
| Lutensol ® M 7 | Nonionic Surfactant | C10C18-Alcohol + 7 EO |
| Lutensol ® ON 30 | Nonionic Surfactant | C10-Oxo alcohol + 3 EO |
| Lutensol ® ON 50 | Nonionic Surfactant | C10-Oxo alcohol + 5 EO |
| Lutensol ® ON 70 | Nonionic Surfactant | C10-Oxo alcohol + 7 EO |
| Lutensol ® ON 80 | Nonionic Surfactant | C10-Oxo alcohol + 8 EO |
| Lutensol ® PO 5 | Emulsifier and | C8C10-Fatty alcohol + approx. 5 EO |
| | Solubilizers | |
| Lutensol ® TO 10 | Nonionic Surfactant | C13-Oxo alcohol + 10 EO |
| Lutensol ® TO 12 | Nonionic Surfactant | C13-Oxo alcohol + 12 EO |
| Lutensol ® TO 129 | Nonionic Surfactant | C13-Oxo alcohol + 12 EO |
| Lutensol ® TO 2 | Nonionic Surfactant | C13-Oxo alcohol + 2 EO |
| Lutensol ® TO 20 | Nonionic Surfactant | C13-Oxo alcohol + 20 EO |
| Lutensol ® TO 3 | Nonionic Surfactant | C13-Oxo alcohol + 3 EO |
| Lutensol ® TO 389 | Nonionic Surfactant | C13-Oxo alcohol + 3 EO/+8 EO |
| Lutensol ® TO 5 | Nonionic Surfactant | C13-Oxo alcohol + 5 EO |
| Lutensol ® TO 6 | Nonionic Surfactant | C13-Oxo alcohol + 6 EO |
| Lutensol ® TO 7 | Nonionic Surfactant | C13-Oxo alcohol + 7 EO |
| Lutensol ® TO 8 | Nonionic Surfactant | C13-Oxo alcohol + 8 EO |
| Lutensol ® XL 100 | Nonionic Surfactant | C10-Guerbet alcohol alkoxylate + 10 |
| | | EO |
| Lutensol ® XL 140 | Nonionic Surfactant | C10-Guerbet alcohol alkoxylate + 14 |
| | | EO |
| Lutensol ® XL 40 | Nonionic Surfactant | C10-Guerbet alcohol alkoxylate + 4 EO |
| Lutensol ® XL 50 | Nonionic Surfactant | C10-Guerbet alcohol alkoxylate + 5 EO |
| Lutensol ® XL 60 | Nonionic Surfactant | C10-Guerbet alcohol alkoxylate + 6 EO |
| Lutensol ® XL 70 | Nonionic Surfactant | C10-Guerbet alcohol alkoxylate + 7 EO |
| Lutensol ® XL 80 | Nonionic Surfactant | C10-Guerbet alcohol alkoxylate + 8 EO |
| Lutensol ® XL 90 | Nonionic Surfactant | C10-Guerbet alcohol alkoxylate + 9 EO |
| Lutensol ® XP 100 | Nonionic Surfactant | C10-Guerbet alcohol alkoxylate + 10 |
| | | EO |
| Lutensol ® XP 30 | Nonionic Surfactant | C10-Guerbet alcohol alkoxylate + 3 EO |
| Lutensol ® XP 40 | Nonionic Surfactant | C10-Guerbet alcohol alkoxylate + 4 EO |
| Lutensol ® XP 50 | Nonionic Surfactant | C10-Guerbet alcohol alkoxylate + 5 EO |
| Lutensol ® XP 60 | Nonionic Surfactant | C10-Guerbet alcohol alkoxylate + 6 EO |
| Lutensol ® XP 70 | Nonionic Surfactant | C10-Guerbet alcohol alkoxylate + 7 EO |
| Lutensol ® XP 80 | Nonionic Surfactant | C10-Guerbet alcohol alkoxylate + 8 EO |
| Lutensol ® XP 90 | Nonionic Surfactant | C10-Guerbet alcohol alkoxylate + 9 EO |
| Monophenylglycol | Emulsifiers and | Phenoxyethanol |
| | Solubilizers | |
| Phenoxypropanol | Emulsifiers and | Phenoxypropanol |
| | Solubilizers | |

The below listed Plurafac ® LF type produicts are low-foaming non-ionic surfactants. They
consist of alkoyxylated, predominantly unbranched fatty alcohols and contain higher
alkene oxides alongside ethylene oxide.
The fatty alcohol chains of Plurafac@ LF 131, 132, 231, and 431 terminate in an alkyl
group.
Their most important properties are wetting, low-foaming or even defoaming.

| | | |
|---|---|---|
| Plurafac ® LF 120 | Low-foaming Nonionic | Fatty alcohol alkoxylate |
| | Surfactants | |
| Plurafac ® LF 1300 | Low-foaming Nonionic | Fatty alcohol alkoxylate |
| | Surfactants | |
| Plurafac ® LF 131 | Low-foaming Nonionic | Fatty alcohol alkoxylate, end-capped |
| | Surfactants | |
| Plurafac ® LF 1430 | Low-foaming Nonionic | Amine alkoxylate |
| | Surfactants | |
| Plurafac ® LF 220 | Low-foaming Nonionic | Fatty alcohol alkoxylate |
| | Surfactants | |
| Plurafac ® LF 221 | Low-foaming Nonionic | Fatty alcohol alkoxylate |
| | Surfactants | |
| Plurafac ® LF 223 | Low-foaming Nonionic | Fatty alcohol alkoxylate |
| | Surfactants | |
| Plurafac ® LF 224 | Low-foaming Nonionic | Fatty alcohol alkoxylate |
| | Surfactants | |
| Plurafac ® LF 231 | Low-foaming Nonionic | Fatty alcohol alkoxylate, end-capped |
| | Surfactants | |
| Plurafac ® LF 300 | Low-foaming Nonionic | Fatty alcohol alkoxylate |
| | Surfactants | |
| Plurafac ® LF 303 | Low-foaming Nonionic | Fatty alcohol alkoxylate |
| | Surfactants | |
| Plurafac ® LF 305 | Low-foaming Nonionic | Fatty alcohol alkoxylate |
| | Surfactants | |
| Plurafac ® LF 400 | Low-foaming Nonionic | Fatty alcohol alkoxylate |
| | Surfactants | |
| Plurafac ® LF 401 | Low-foaming Nonionic | Fatty alcohol alkoxylate |
| | Surfactants | |
| Plurafac ® LF 403 | Low-foaming Nonionic | Fatty alcohol alkoxylate |
| | Surfactants | |
| Plurafac ® LF 404 | Low-foaming Nonionic | Fatty alcohol alkoxylate |
| | Surfactants | |
| Plurafac ® LF 431 | Low-foaming Nonionic | Fatty alcohol alkoxylate, end-capped |
| | Surfactants | |
| Plurafac ® LF 500 | Low-foaming Nonionic | Fatty alcohol alkoxylate |
| | Surfactants | |
| Plurafac ® LF 600 | Low-foaming Nonionic | Fatty alcohol alkoxylate |
| | Surfactants | |
| Plurafac ® LF 711 | Low-foaming Nonionic | Fatty alcohol alkoxylate |
| | Surfactants | |
| Plurafac ® LF 7319 | Low-foaming Nonionic | Fatty alcohol alkoxylate, modified |
| | Surfactants | |
| Plurafac ® LF 801 | Low-foaming Nonionic | Fatty alcohol alkoxylate |
| | Surfactants | |
| Plurafac ® LF 802 | Low-foaming Nonionic | Fatty alcohol alkoxylate |
| | Surfactants | |
| Plurafac ® LF 900 | Low-foaming Nonionic | Fatty alcohol alkoxylate |
| | Surfactants | |
| Plurafac ® LF 901 | Low-foaming Nonionic | Fatty alcohol alkoxylate |
| | Surfactants | |
| Plurafac ® SLF 180 | Low-foaming Nonionic | Fatty alcohol alkoxylate |
| | Surfactants | |
| Pluronic ® PE 6100 | Low-foaming Nonionic | PO-EO-block polymer - approx. 10% |
| | Surfactants | EO |
| Pluronic ® PE 6200 | Low-foaming Nonionic | PO-EO-block polymer - approx. 20% |
| | Surfactants | EO |
| Pluronic ® PE 6400 | Low-foaming Nonionic | PO-EO-block polymer - approx. 40% |
| | Surfactants | EO |
| Pluronic ® PE 8100 | Low-foaming Nonionic | PO-EO-block polymer - approx. 10% |
| | Surfactants | EO |
| Pluronic ® RPE 1720 | Low-foaming Nonionic | EO-PO-block polymer - approx. 20% |
| | Surfactants | EO |
| Pluronic ® RPE 1740 | Low-foaming Nonionic | EO-PO-block polymer - approx. 40% |
| | Surfactants | EO |
| Pluronic ® RPE 2520 | Low-foaming Nonionic | EO-PO-block polymer - approx. 20% |
| | Surfactants | EO |
| Pluronic ® RPE 3110 | Low-foaming Nonionic | EO-PO-block polymer - approx. 10% |
| | Surfactants | EO |

### Amphoteric Surfactants.

Amphoteric surfactants include water-soluble amine oxides containing one alkyl moiety of from about 8 to about 18 carbon atoms and 2 moieties selected from the group consisting of alkyl moieties and hydroxyalkyl moieties containing from about 1 to about 3 carbon atoms, water-soluble sulfoxides containing one alkyl moiety of from about 10 to about 18 carbon atoms and a moiety selected from the group consisting of alkyl moieties and hydroxy-alkyl moieties of from about 1 to about 3 carbon atoms, amine oxides, such as lauryl dimethyl amine oxide (“lauramine oxide”). Amine oxides include alkyl dimethyl amine oxides, alkyl amido propyl dimethyl amine oxides, alkyl dimethyl amine oxides, coco dimethyl amino oxides. Amine oxides may have a linear or mid-branched alkyl moiety. Typical linear amine oxides include water-soluble amine oxides containing one R1=C₈₋₁₈ alkyl moiety and two R² and R³ moieties selected from the group consisting of C₁-C₃ alkyl groups and C₁-C₃ hydroxyalkyl groups. In one embodiment, the amine oxide is characterized by the formula

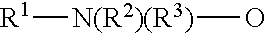

wherein R¹ is a C₈₋₁₈ alkyl and R² and R³ are selected from the group consisting of methyl, ethyl, propyl, isopropyl, 2-hydroxethyl, 2-hydroxypropyl and 3-hydroxypropyl. The linear amine oxide surfactants include linear C₁₀-C₁₈ alkyl dimethyl amine oxides and linear C₈-C₁₂ alkoxy ethyl dihydroxy ethyl amine oxides. Amine oxides include linear C₁₀, linear C₁₀-C₁₂, and linear C₁₂-C₁₄ alkyl dimethyl amine oxides. As used herein “mid-branched” means that the amine oxide has one alkyl moiety having n1 carbon atoms with one alkyl branch on the alkyl moiety having n2 carbon atoms. The alkyl branch is located on the alpha carbon from the nitrogen on the alkyl moiety. This type of branching for the amine oxide is also known in the art as an internal amine oxide. The total sum of n1 and n2 is from 10 to 24 carbon atoms, preferably from 12 to 20, and more preferably from 10 to 16. The number of carbon atoms for the one alkyl moiety (n1) should be approximately the same number of carbon atoms as the one alkyl branch (n2) such that the one alkyl moiety and the one alkyl branch are symmetric. As used herein “symmetric” means that (n1-n2) is less than or equal to 5, preferably 4, most preferably from 0 to 4 carbon atoms in at least 50 wt. %, more preferably at least 75 wt. % to 100 wt. % of the mid-branched amine oxides. The amine oxide further comprises two moieties, independently selected from a C₁-C₃ alkyl, a C₁-C₃ hydroxyalkyl group, or a polyethylene oxide group containing an average of from about 1 to about 3 ethylene oxide groups. Preferably the two moieties are selected from a C₁-C₃ alkyl, more preferably both are selected as a C₁ alkyl. Amphoteric surfactants include C₈-C₁₈ alkyl-dimethyl aminoxides and C₈-C₁₈ alkyl-di(hydroxyethyl)aminoxides.

### Zwitterionic Surfactants.

Zwitterionic surfactants include betaines, such as alkyl betaines, alkylamidobetaine, amidazolin-iumbetaine, sulfobetaine (INCI Sultaines) as well as phosphobetaines.

Examples of betaines and sulfobetaines are the following (designated in accordance with INCI): Almond amidopropyl of betaines, Apricotamidopropyl betaines, Avocadamidopropyl of betaines, Babassuamidopropyl of betaines, Behenamidopropyl betaines, Behenyl of betaines, Canol amidopropyl betaines, Capryl/Capramidopropyl betaines, Carnitine, Cetyl of betaines, Cocamidoethyl of betaines, Cocamidopropyl betaines, Cocamidopropyl Hydroxysultaine, Coco betaines, Coco Hydroxysultaine, Coco/Oleam idopropyl betaines, Coco Sultaine, Decyl of betaines, Dihy-droxyethyl Oleyl Glycinate, Dihydroxyethyl Soy Glycinate, Dihydroxyethyl Stearyl Glycinate, Dihydroxyethyl Tallow Glycinate, Dimethicone Propyl of PG-betaines, Erucamidopropyl Hy-droxysultaine, Hydrogenated Tallow of betaines, Isostearamidopropyl betaines, Lauramidopro-pyl betaines, Lauryl of betaines, Lauryl Hydroxysultaine, Lauryl Sultaine, Milkamidopropyl beta-ines, Minkamidopropyl of betaines, Myristamidopropyl betaines, Myristyl of betaines, Oleami-dopropyl betaines, Oleamidopropyl Hydroxysultaine, Oleyl of betaines, Olivamidopropyl of beta-ines, Palmamidopropyl betaines, Palmitamidopropyl betaines, Palmitoyl Carnitine, Palm Ker-nelamidopropyl betaines, Polytetrafluoroethylene Acetoxypropyl of betaines, Ricinoleam idopro-pyl betaines, Sesamidopropyl betaines, Soyamidopropyl betaines, Stearamidopropyl betaines, Stearyl of betaines, Tallowamidopropyl betaines, Tallowamidopropyl Hydroxysultaine, Tallow of betaines, Tallow Dihydroxyethyl of betaines, Undecylenamidopropyl betaines and Wheat Ger-mamidopropyl betaines.

Examples of amphoteric surfactants are Cocoamidopropyl betaine (e.g. Dehyton® K, Dehyton® KE UP, Dehyton@ PK 45, Dehyton® P CAW) and N,N-Dimethyldodecan-1-amine N-oxide (Lauramine oxide, e.g. Dehyton® PL)

### Cationic Surfactants

Surfactants comprise include cationic surfactants. Cationic surfactants include quaternary ammonium surfactants, with up to 26 carbon atoms, as for instance alkoxylated quaternary ammonium (AQA) surfactants as discussed in U.S. Pat. No. 6,136,769, dimethyl hydroxyethyl quaternary ammonium as discussed in U.S. Pat. No. 6,004,922, dimethyl hydroxyethyl lauryl ammonium chloride, polyamine cationic surfactants as discussed in WO 98/35002, WO 98/35003, WO 98/35004, WO 98/35005, and WO 98/35006, cationic ester surfactants as discussed in U.S. Pat. Nos. 4,228,042, 4,239,660 4,260,529 and U.S. Pat. No. 6,022,844, and amino surfactants as discussed in U.S. Pat. No. 6,221,825 and WO 00/47708, specifically amido propyldimethyl amine (APA).

Surfactants further comprise isoprenoid surfactants represented by formula A and formula B

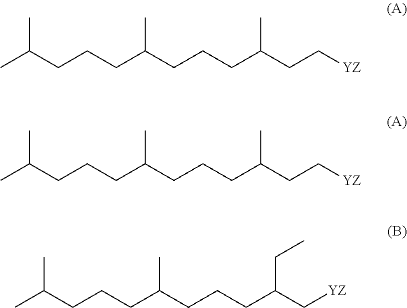

where Y is CH₂ or null, and Z may be chosen such that the resulting surfactant is selected from the following surfactants: an alkyl carboxylate surfactant, an alkyl polyalkoxy surfactant, an alkyl anionic polyalkoxy sulfate surfactant, an alkyl glycerol ester sulfonate surfactant, an alkyl dimethyl amine oxide surfactant, an alkyl polyhydroxy based surfactant, an alkyl phosphate ester surfactant, an alkyl glycerol sulfonate surfactant, an alkyl polygluconate surfactant, an alkyl polyphosphate ester surfactant, an alkyl phosphonate surfactant, an alkyl polyglycoside surfactant, an alkyl monoglycoside surfactant, an alkyl diglycoside surfactant, an alkyl sulfosuccinate surfactant, an alkyl disulfate surfactant, an alkyl disulfonate surfactant, an alkyl sulfosuccinamate surfactant, an alkyl glucamide surfactant, an alkyl taurinate surfactant, an alkyl sarcosinate surfactant, an alkyl glycinate surfactant, an alkyl isethionate surfactant, an alkyl dialkanolamide surfactant, an alkyl monoalkanolamide surfactant, an alkyl monoalkanolamide sulfate surfactant, an alkyl diglycolamide surfactant, an alkyl diglycolamide sulfate surfactant, analkyl glycerol ester surfactant, an alkyl glycerol ester sulfate surfactant, an alkyl glycerol ether surfactant, an alkyl glycerol ether sulfate surfactant, alkyl methyl ester sulfonate surfactant, an alkyl polyglycerol ether surfactant, an alkyl polyglycerol ether sulfate surfactant, an alkyl sorbitan ester surfactant, an alkyl ammonioalkanesulfonate surfactant, an alkyl amidopropyl betaine surfactant, an alkyl allylated quat based surfactant, an alkyl monohydroxyalkyl-di-alkylated quat based surfactant, an alkyl di-hydroxyalkyl monoalkyl quat based surfactant, an alkylated quat surfactant, an alkyl trimethylammonium quat surfactant, an alkyl polyhydroxalkyl oxypropyl quat based surfactant, an alkyl glycerol ester quat surfactant, an alkyl glycol amine quat surfactant, an alkyl monomethyl dihydroxyethyl quaternary ammonium surfactant, an alkyl dimethyl monohydroxyethyl quaternary ammonium surfactant, an alkyl trimethylammonium surfactant, an alkyl imidazolinebased surfactant, an alken-2-yl-succinate surfactant, an alkyl a-sulfonated carboxylic acid surfactant, an alkyl a-sulfonated carboxylic acid alkyl ester surfactant, an alpha olefin sulfonate surfactant, an alkyl phenol ethoxylate surfactant, an alkyl benzenesulfonate surfactant, an alkyl sulfobetaine surfactant, an alkyl hydroxysulfobetaine surfactant, an alkyl ammoniocarboxylate betaine surfactant, an alkyl sucrose ester surfactant, an alkyl alkanolamide surfactant, an alkyl di(polyoxyethylene) monoalkyl ammonium surfactant, an alkyl mono(polyoxyethylene)dialkyl ammonium surfactant, an alkyl benzyl dimethylammonium surfactant, an alkyl aminopropionate surfactant, an alkyl amidopropyl dimethylamine surfactant, or a mixture thereof; and if Z is a charged moiety, Z is charge-balanced by a suitable metal or organic counter ion. Suitable counter ions include a metal counter ion, an amine, or an alkanolamine, e.g., C₁-C₆ alkanolammonium. More specifically, suitable counter ions include Na+, Ca+, Li+, K+, Mg+, e.g., monoethanolamine (MEA), diethanolamine (DEA), triethanolamine (TEA), 2-amino-1-propanol, 1-aminopropanol, methyldiethanolamine, dimethylethanolamine, monoisopropanolamine, triisopropanolamine, 1-amino-3-propanol.

### Surface Care Agents

Care composition ingredients according to this invention include surface care agents. Surface care agents include, without being limited to, the compounds listed in Table “Surface care agents” below.

### Surface Care Agents

| Trade name | Chemical class |
|---|---|
| Polyquart ® 149 A | Acrylic copolymer, sodium salt |
| Polyquart ® Ecoclean | Amphoteric modified starch |
| Polyquart ® PN 60 | Polyethylene imine, modified |
| Polyquart ® Pro A | Acrylic copolymer, sodium salt |
| Polyquart ® S | Amphoteric modified starch |
| Polyquart ® SD 09 | Cationic polymer |

### Polyalkylene Glycols

Care composition ingredients according to this invention include polyalkylene glycols. Depending on their molar mass, polyalkylene glycols like the ones commercially available as Pluriol® E grades and Pluriol® P grades can be used as solubilizers, plasticizers, binders, foam suppressors, humectants, lubricants, dispersing agents, and mould-release agents.

### UV Light Stabilizers

Care composition ingredients according to this invention include UV light stabilizers. UV light stabilizers include Sodium 3-(2H-benzotriazol-2-yl)-5-sec-butyl-4-hydroxybenzenesulfonate (e.g. Cibafast® HA Liquid), 2-(2H-benzotriazol-2-yl)-6-dodecyl-4-methyl-phenol and 2-ethoxyethyl(2Z)-2-cyano-2-[3-(3-methoxypropylamino)cyclohex-2-en-1-ylidene] acetate (e.g. Tinogard® CT), Benzenesulfonic acid, 3-(2H-benzotriazol-2-yl)-4-hydroxy-5-(1-methylpropyl)-, monosodium salt (e.g. Tinogard® HS), and 2-(2H-benzotriazol-2 yl)-6-dodecyl-4-methyl-phenol (e.g. Tinogard® TL).

### Waxes and Emulsions

Care composition ingredients according to this invention include waxes. Waxes include, without being limited to, polyvinylethers, oxidized polyethylenes (PE), and ethylene copolymers. Examples of waxes are the ones listed in the table “Waxes” below.

### Table “Waxes”

| | | Chemical |
|---|---|---|
| Trade name | Application | constitution |
| Poligen ® Wax V | Surface protection, floor polishes, | Polyvinyl |
| | leather polishes, car polishes | ether wax |
| Poligen ® WE 1 | Hard Surface Cleaning, Institutional | Oxidized PE |
| | Cleaning and Sanitation, Vehicle and | hard wax |
| | Transportation Care, | |
| | Industrial Cleaning | |
| Poligen ® WE 3 | Hard Surface Cleaning, Institutional | Ethylene |
| | Cleaning and Sanitation, Vehicle and | copolymer |
| | Transportation Care, | wax |
| | Industrial Cleaning | |
| Poligen ® WE 4 | Hard Surface Cleaning, Institutional | Ethylene |
| | Cleaning and Sanitation, Vehicle and | copolymer |
| | Transportation Care, | wax |
| | Industrial Cleaning | |
| Poligen ® WE 6 | Hard Surface Cleaning, Institutional | Oxidized |
| | Cleaning and Sanitation, Vehicle and | polyethylene |
| | Transportation Care, | wax |
| | Industrial Cleaning | |

The chemical production network may include one or more one or more chemical and/or mechanical process(es). The chemical production network may produce one or more output material(s) through chemical and/or mechanical processing. The chemical production network may include multiple types of production processes for producing one or more output material(s) from one or more input material(s). The chemical production network may produce one or more output material(s) from input material(s) provided to the chemical production network. The chemical production network may include a complex production network producing multiple chemical products via multiple production process(es). The chemical production network may include connected, interconnected and/or non-connected production process(es). The chemical production network may include a composite or Verbund network.

The chemical production network may include identity preserving or segregated production process(es). Identity preserving or segregated in this context may refer to environmental attribute(s) of input material(s) being preserved or segregated in the production process(es). Examples are non-fossil, e.g. renewable or recycled, input materials used to produce the one or more care composition ingredient(s) without fossil content. Further examples are fossil input material(s) used to produce the one or more care composition ingredient(s) with fossil content. Chemical production networks may include non-identity preserving or non-segregated production process(es). Non-identity preserving or non-segregated in this context may refer to non-fossil input material(s) being mixed with fossil input material(s) to produce the care composition ingredient(s). For example, fossil and renewable input materials may be mixed to produce the care composition ingredient(s) with fossil and renewable content.

The chemical production network may include one or more production process(es) with multiple production steps. The production steps included in the chemical network may be defined by the physical system boundary of the chemical production network. The system boundary may be defined by location and/or control over production processes or steps. The system boundary may be defined by a site of the chemical production network. The system boundary may be defined by production process(es) or step(s) controlled by one entity or multiple entities jointly. The system boundary may be defined by the value chain with staggered production process(es) or step(s) to the chemical end product, which may be controlled by multiple entities jointly or separately. The chemical production network may include a waste collection, a sorting step, a recycling step such as pyrolysis, a cracking step such as steam cracking, a separation step to separate intermediates of one process step and further processing steps to convert such intermediates to output material(s), in particular the produced care composition ingredient(s) leaving the system boundary of the chemical production network. The input material(s) may enter the physical system boundary of the chemical production network. The entry point(s) of the chemical production network may be marked by the entry of input material(s) to the chemical production network or the system boundary of the chemical network. The output material(s), in particular the produced care composition ingredient(s), may leave the physical system boundary of the chemical production network. The exit point(s) of the chemical production network may be marked by the exit of output material(s), in particular the produced care composition ingredient(s), from the chemical production network or the system boundary of the chemical network.

The chemical production network may include one or more production chain(s) for the production of care composition ingredients. The production chain(s) for the production of care composition ingredients may be interconnected. The production chain(s) for the production of care composition ingredients may be interconnected with production chain(s) for the production of other output material(s). The production chain(s) for the production of care composition ingredients may include production chain(s) for the production of intermediates used to produce care composition ingredients. The production chain(s) for the production of care composition ingredients may use input material(s) provided by chemical network(s) for the production of intermediates usable to produce care composition ingredients.

One or more input material(s) may be provided to the chemical production network for producing one or more output material(s), in particular care composition ingredient(s). The output material(s), in particular care composition ingredient(s), may be produced from one or more input material(s) through one or more chemical process(es) of the chemical production network. The input material may comprise any input material entering the chemical production network at any entry point. The input material may include organic or inorganic input material(s). The input material may be a pre-cursor product, an intermediate material, a feedstock or a raw material used to produce one or more output material(s), in particular care composition ingredient(s). The input material may be fed to the chemical production network to produce one or more output material(s), in particular care composition ingredient(s). The input material may be fed to chemical production network including one or more production process(es) with multiple process steps. The input material may be fed to the chemical production network at the start of the production process or at any intermediate stage of the production process. The input materials entering the chemical production network may be used to produce one or more output material(s), in particular care composition ingredient(s).

The input material may be associated with an input material identifier. The input material identifier may comprise any identifier uniquely associated with the input material. The input material identifier may be associated with the physical entity of the input material. The input material identifier may be associated with a single batch of input material. The input material identifier may be associated with a group of input materials. The identifier may be associated with multiple physical entities of the input material. The input material identifier may be associated with continuous or semi-continuous stream of input material. The input material identifier may be associated with a stream of the input material e.g. over a certain time period or from a certain supplier. The input material identifier may be linked or connected to one or more environmental attribute(s).

Environmental attribute may refer to a property related to the environmental impact. Such property may be the property of input material(s), chemical process(es), chemical production network(s) and/or care composition ingredient(s). The environmental attribute may indicate an environmental performance of input material(s), chemical process(es), chemical production network(s) and/or care composition ingredient(s). The environmental attribute may be derived from properties of input material(s), chemical process(es), chemical production network(s) and/or care composition ingredient(s). The environmental attribute may be associated with the environmental impact of input material(s), chemical process(es), chemical production network(s) and/or care composition ingredient(s) at any stage of the lifecycle of the input material(s) and/or care composition ingredient(s). The stages may include providing raw material, providing feedstock, producing chemical products, such as intermediate products or end products, producing discrete products by using the chemical products, using chemical products or discrete products, treating end-of-life products, recycling end-of-life products, disposing end-of-life products, reusing components from end-of-life products or any subset of stages. The environmental attribute may be specified or may be derived from any activity of one or more entities participating at any stage of the lifecycle of one or more material(s) or product(s).

The environmental attribute may include one or more characteristic(s) that are attributable to environmental impact of input material(s), chemical process(es), chemical production network(s) and/or care composition ingredient(s). The environmental attribute may include environmental, technical, recyclability or circularity characteristics(s) associated with the environmental impact of input material(s), chemical process(es), chemical production network(s) and/or care composition ingredient(s).

The one or more environmental attribute(s) may be attributable to the environmental impact of the care composition ingredient. The one or more environmental attribute(s) may relate to environmental, technical, recyclability, circularity and/or complementary risk characteristic(s) of the care composition ingredient.

Environmental characteristic(s) may specify or quantify ecological criteria associated with environmental impact. Environmental characteristic(s) may be or may be derived from measurements taken during the lifecycle. Environmental characteristics may be determined at any stage of the lifecycle and may characterize the environmental impact for such stage or up to such stage. Environmental characteristic(s) may for example include carbon footprint, greenhouse gas emissions, resource usage, air emissions, ozone depletion potential, water pollution, noise pollution, energy consumption, waste reduction, or eutrophication potential. Environmental characteristic(s) may for example include product characteristics related to the production of the product like bio based, vegetable based, animal based, halogen-free, fluorine-free, vegan, halal, kosher, palm oil-free, natural, tox-fee, volatile organic compounds-free or any combinations thereof.

Technical characteristic(s) may specify or quantify performance at least indirectly associated with the environmental impact. Technical characteristic(s) may be or may be derived from measurements taken during the lifecycle. Technical characteristics may be determined at any stage of the lifecycle and may characterize the performance for such stage or up to such stage. Technical characteristic(s) may for example include chemical composition data, raw material composition such as bio-based or recycled input material content specifying e.g. x % non fossil and y % fossil content, bill of materials, product specification data such as product purity, product form (as indication to their impact on dust formation/release), safety data, product extractability, migration data, toxicological data or ecotoxicological data, product component data, safety data, application property data, application instructions, quality data or any combinations thereof.

Circularity characteristic(s) may specify or quantify the life cycle characteristics associated with circular uses. Circularity characteristic(s) may be or may be derived from measurements taken during the lifecycle. Circularity characteristic(s) may be or may be derived from circular data recorded in one or more prior lifecycle(s) including reuse. Circularity characteristic(s) may be determined at any stage of the lifecycle and may characterize the reuse or recycling performance for such stage or up to such stage. Circularity characteristic(s) may for example include recycling data, reuse rate, recycling rate, recycling loops, reuse performance, reused quality or any combinations thereof.

Recyclability characteristic(s) may specify or quantify life cycle characteristics associated with recycling uses. Recyclability characteristic(s) may include the composition of the material includeing specifically tailored constituents making the material suitable for recycling. Recyclability characteristic(s) may be or may be derived from measurements taken during the lifecycle. Recyclability characteristic(s) may be or may be derived from recycling data recorded in one or more prior lifecycle(s). Recyclability characteristics may be determined at any stage of the lifecycle and may characterize the recycling performance for such stage or up to such stage. Recyclability characteristic(s) may for example include recycling data, number of reuses, recyclate composition, recyclate quality, waste stream composition, waste stream quality or any combinations thereof.

In one embodiment the care composition ingredients passport or digital asset associated with the care composition ingredient may include mass balanced environmental attributes related to the input material. Mass balanced environmental attributes may include environmental attributes of the input material(s) used to produce the care composition ingredient, which are tracked and by mass attributable to the care composition ingredient. The environmental impact of input material(s) may be determined based on input material(s) used in the chemical process(es) to produce the care composition ingredient. For example, bio-based, renewable and/or recycled content of input material(s) used to produce the care composition ingredient may be tracked. Further for example, castor oil or palm oil content of input material(s) used to produce the care composition ingredient may be tracked. Further for example, properties related to the environmental impact of the input material include RSPO palm oil, palm oil free or castor oil may be tracked. Further for example, properties of the chemical process(es) used to produce the care composition ingredient may be tracked. Examples of tracked process properties related to the environmental impact include water consumption, CO₂ emissions and/or Greenhouse Gas (GHG) emissions, amount of waste generation, mixed material generation, design for recycling, energy consumption, processing properties such as less waste or less loss of properties. The properties may be tracked based on a certificate from a certifying agency. The properties may be tracked based on inherent physical properties derived from measurements.

In one embodiment the produced care composition ingredient is connected to the decentral identifier physically identifying the produced care composition ingredient. The production operating apparatus may be configured to provide the decentral identifier associated with a physical entity of the produced care composition ingredient. The production operating apparatus may be configured to link the decentral identifier to a physical identifier of the produced care composition ingredient. The production operating apparatus may be configured to assign the decentral identifier to the physical identifier connected to the produced care composition ingredient. The production operating apparatus may be configured to assign the decentral identifier to the physical identifier physically connected to the produced care composition ingredient.

In one embodiment the decentral identifier relates to data associated with at least one product produced from the care composition ingredient, wherein the one or more environmental attribute(s) associated with the at least one product is derived from one or more environmental attribute(s) associated with the care composition ingredient. The one or more environmental attribute(s) associated with the care composition ingredient may be associated with the one or more input material(s) and/or the chemical process(es) used to produce the care composition ingredient. The decentral identifier may relate to any identifier uniquely associated with the care composition ingredient. The decentral identifier may be associated with the physical entity of the care composition ingredient. The decentral identifier may refer to a single batch of care composition ingredient. The decentral identifier may be associated with a group of care composition ingredients. The identifier may refer to multiple physical entities of the care composition ingredient. The decentral identifier may be associated with continuous or semi-continuous stream of care composition ingredient. The identifier may refer to a stream of the care composition ingredient e.g. over a certain time period or from a certain supplier.

In one embodiment the one or more environmental attribute(s) associated with the care composition ingredient(s) are provided from at least one balancing account configured to store environmental attribute(s) associated with input material(s). The balancing account may relate to storage structure associated with metadata, such as an environmental attribute type. For instance, the balancing account may be associated with metadata indicating the environmental attribute type to be recycled-content or bio-based. An inbound allocator may be configured to allocate the one or more environmental attribute(s) associated with input material(s) to at least one balancing account e.g. on entry of the input material to the chemical production network. The balancing account may be associated with the respective environmental attribute type. An outbound assigner may be configured to assign at least one environmental attribute from the at least one balancing account associated with the respective environmental attribute to the decentral identifier. One or more environmental attribute(s) may be assigned to the at least one decentral identifier. Assignment may include de-allocation of the one or more environmental attributes from the balancing account associated with the respective environmental attribute type. By using the balancing accounts environmental attributes of input materials can be reliably tracked and assigned to care composition ingredients.

In one embodiment the one or more environmental attribute(s) associated with the care composition ingredient(s) are provided from at least one balancing account configured to store environmental attribute(s) associated with input material(s). An inbound allocator may be configured to allocate the one or more environmental attribute(s) to at least one balancing account associated with the respective environmental attribute, e.g. on input material entering the chemical production network. The balancing account may be associated with the respective environmental attribute type. The one or more environmental attribute(s) associated with the input materials may be allocated to the balancing account associated with the respective environmental attribute type. An outbound assigner may be configured to assign or link at least one environmental attribute from the at least one balancing account associated with the respective environmental attribute type to the decentral identifier. This may include de-allocation of the one or more environmental attribute(es) from the balancing account associated with the respective environmental attribute type. By using the balancing accounts environmental attributes can be tacked through the chemical production network. This way the environmental attributes may be detached from the material flow. Attribution of environmental attribute(s) may be conducted on a mass balance basis for the chemical production network. In such approach the total mass of input materials and care composition ingredients as well as the attribution of respective environmental attribute(s) associated with input materials and care composition ingredients are balanced.

In one embodiment the one or more environmental attribute(s) are associated with at least one property related to the environmental impact of the one or more input material(s) and/or the chemical process(es). The one or more environmental attribute(s) may specify environmental properties of the input material(s) used to produce the care composition ingredient and/or the one or more environmental attribute(s) may specify environmental properties of the chemical process(es) used to produce the care composition ingredient. The one or more environmental attribute(s) may be generated from environmental properties of the input material(s) used to produce the care composition ingredient, process data associated with the chemical processing of the input material(s) and/or energy data associated with the energy consumption of the chemical processing. The one or more environmental attribute(s) may include a recycled content associated with the input material(s) and allocated or allocatable to the care composition ingredient(s), a renewable content associated with the input material(s) and allocated or allocatable to the care composition ingredient(s), and/or a product carbon footprint associated with the care composition ingredient(s).

In one embodiment the production operating apparatus is configured to gather environmental attributes associated with the produced care composition ingredient before, during and/or after production of the care composition ingredient by the chemical production network. The environmental attributes associated with the produced care composition ingredient may relate to input material(s). The environmental attributes associated with input materials may be provided before, during and/or after production of the care composition ingredient by the chemical production network. The environmental attributes associated with input materials may be allocated to at least one balancing account before, during and/or after production of the care composition ingredient by the chemical production network. The environmental attributes associated with the produced care composition ingredient may relate to environmental properties generated from process data associated with the chemical processing of the input material(s) and/or energy data associated with the energy consumption of the chemical processing. The environmental attributes associated with the produced care composition ingredient may be generated before, during and/or after production of the care composition ingredient by the chemical production network. The process data associated with the chemical processing of the input material(s) and/or energy data associated with the energy consumption of the chemical processing may be gathered prior, during and/or after production of the care composition ingredient.

In one embodiment the care composition ingredient passport or the digital asset may include the decentral identifier associated with the care composition ingredient and the one or more environmental attribute(s) linked to the decentral identifier. The one or more environmental attribute(s) may be linked to the decentral identifier included in the care composition ingredient passport or the digital asset. The one or more environmental attribute(s) may be stored in a data base associated with or of the care composition ingredient producer for access by any consumer of the care composition ingredient. The one or more environmental attribute(s) may be stored in a data base associated with or of the care composition ingredient producer for transfer to a consume of the care composition ingredient e.g. when accessed or on providing the care composition ingredient. The decentral identifier may comprise any unique identifier uniquely associated with the care composition ingredient producer and care composition ingredient data such as the environmental attributes. The decentral identifier may include a Universally Unique IDentifier (UUID) or a Digital IDentifier (DID). The decentral identifier may be issued by a central or decentral identity issuer. The decentral identifier may be linked to authentication and/or authorization information. Via the decentral identifier and its unique association with the care composition ingredient producer and care composition ingredient data, such as the environmental attributes, access to the care composition ingredient data may be controlled by the care composition ingredient producer. This contrasts with central authority schemes, where identifiers are provided by such central authority and access to data is controlled by such central authority. Decentral in this context refers to the usage of the identifier in implementation as controlled by the data owner, such as the care composition ingredient producer.

The decentral identifier may be uniquely associated with the care composition ingredient or the physical entity of the care composition ingredient, e.g. as packaged for transportation to the consumer of the care composition ingredient. The decentral identifier may be uniquely to the one or more environmental attribute(s). The care composition ingredient passport or the digital asset may include one or more digital representation(s) pointing to care composition ingredient data including the environmental attribute(s) or parts thereof including the environmental attribute(s). The digital representation may comprise at least one interface to a data providing service. It may further include at least one interface to a data consuming service. It may include an endpoint for data exchange or sharing (resource endpoint) or an endpoint for service interaction (service endpoint), that is uniquely identified via a communication protocol. The digital representation(s) pointing to care composition ingredient data or parts thereof may be uniquely associated with the decentral identifier.

The care composition ingredient passport or the digital asset may comprise or be connected to a digital representation of care composition ingredient data, such as environmental attribute(s). The digital representation may include a representation for accessing the care composition ingredient data, such as environmental attribute(s) or part thereof. The digital representation may include a representation of care composition ingredient data, such as environmental attribute(s). The care composition ingredient passport or the digital asset may include or be connected to data related to the care composition ingredient data, such as environmental attribute(s), the authentication information and the decentral identifier. The data related to the care composition ingredient data, such as environmental attribute(s), may include the digital representation of the care composition ingredient data, such as environmental attribute(s).

### BRIEF DESCRIPTION OF THE DRAWINGS

In the following, the present disclosure is further described with reference to the enclosed figures:

FIG. 1 illustrates schematically an example of a chemical production network producing one or more output material(s) from one or more input material(s) in connection with a production operating system including an attribute management system.

FIG. 2 illustrates schematically an example of attributing environmental attributes of input materials to output materials of the chemical production network.

FIG. 3 illustrates schematically an example of attributing environmental attributes of in-FIG. 3 put materials and chemical processes to an output material of the chemical production network.

FIG. 4 illustrates schematically another example of a method or apparatus for providing environmental attributes associated with output materials to a material user as data consumer via a decentral network.

FIG. 5 illustrates schematically an example of a method or apparatus for providing environmental attributes of output materials across value chains via the decentral network.

FIG. 6 illustrates schematically an example of a chemical production network for producing care composition ingredients associated with the digital asset.

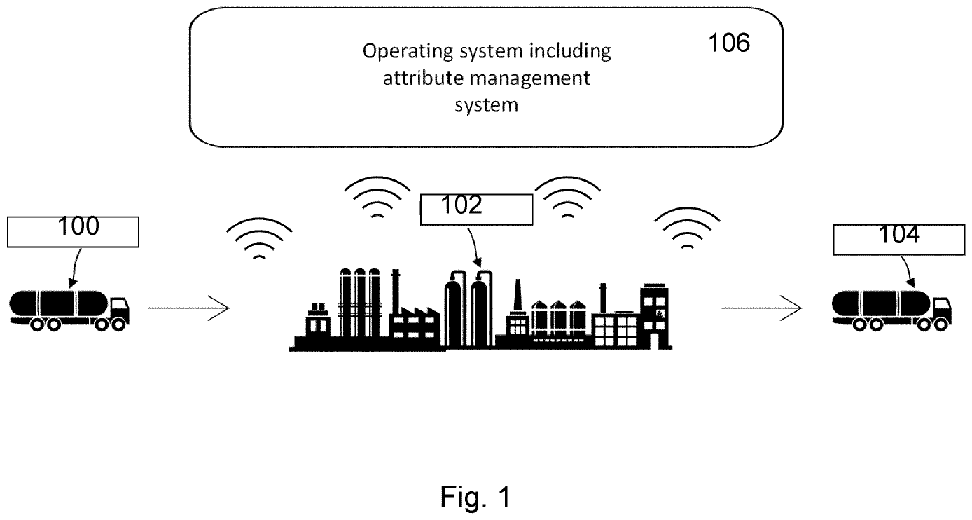

FIG. 1 illustrates an example of a chemical production network 102 producing one or more output material, in particular care composition ingredient(s) 104 from one or more input material(s) 100 in connection with a production operating system 106 including an attribute management system.

For producing one or more output material(s), in particular care composition ingredient(s) 104 different input materials 100 may be provided as physical inputs to the chemical production network 102. The physical input material(s) 100 and output material(s), in particular care composition ingredient(s), 104 may be associated with one or more properties related to environmental impact. The properties related to environmental impact may be digitalized in the form of environmental attributes such as recycled or bio-based content of the input materials. The production operating system 106 may be configured to ingest such environmental attributes and to track the environmental attributes across the chemical production network 102 from input materials 100 to output material(s), in particular care composition ingredient(s), 104.

The chemical production network 102 may include multiple interlinked processing steps. The chemical production network 102 may be an integrated chemical production network 102 with interrelated production chains. The chemical production network 102 may include multiple different production chains that have at least one intermediate product in common. The chemical production network 102 may include multiple stages of the chemical value chain. The chemical production network 102 may include the producing, refining, processing and/or purification of gas or crude oil. The chemical production network 102 may include a stream cracker, or a syngas plant connected to multiple production chains that output products 104 from the effluent of such plants. The chemical production network 102 may include multiple production chains that output from one or more input material(s) 100 one or more output material(s), in particular care composition ingredient(s), 104. The chemical production network 102 may include multiple tiers of a chemical value chain. The chemical production network 102 may include a physically interconnected arrangement of production sites. The production sites may be at the same location or at different locations. In the latter case the production sites may be interconnected by means of dedicated transportation systems such as pipelines, supply chain vehicles, like trucks, supply chain ships or other cargo transportation means.

The chemical production network 102 may chemically convert input materials 100 to one or more output material(s), in particular care composition ingredient(s), 104. The chemical production network 102 may convert input materials 100 by way of chemical conversion to one or more output material(s), in particular care composition ingredient(s), 104.

The input materials 100 may be fed to the chemical production network 102 at any entry point. The input materials 100 may be fed to the chemical production network 102 at the start of the chemical production network 102. Input materials 100 may for example make up the feedstock of a steam cracker. The input material 100 may include non-fossil input material, such as biobased or recycled material, and/or fossil input material for the manufacture of chemical intermediates and chemical output material(s), in particular care composition ingredient(s), 104.

The chemical production network 102 may include multiple production steps. The production steps included in the chemical production network 102 may be defined by the system boundary of the chemical production network 102. The system boundary may be defined by location or control over production processes. The system boundary may be defined by the site of the chemical production network 102. The system boundary may be defined by production processes controlled by one entity or multiple entities jointly. The system boundary may be defined by value chain with staggered production processes to an end product, which may be controlled by multiple entities separately. The chemical production network 102 may include a waste collection and sorting step, a recycling step such as pyrolysis, a cracking step such as steam cracking, a separation step to separate intermediates of one process step and further processing steps to convert such intermediates to output material(s), in particular care composition ingredient(s), 104 leaving the system boundary of the chemical production network 102.

The production operating system 106 of the chemical production network 102 may be configured to monitor and/or control the chemical production network 102 based on operating parameters of the different processes. One process step monitored and/or controlled may be the feed of input materials 100 or the release of output material(s), in particular care composition ingredient(s), 104. Another process step monitored and/or controlled may be the registration of environmental attributes associated with input materials 100 entering the system boundary of the chemical production network 102. Yet another process step monitored and/or controlled may be the attribution of environmental attributes to output material(s), in particular care composition ingredient(s), 104 produced via the chemical production network 102. Yet another process step monitored and/or controlled may be the management of environmental attributes associated with input materials 100 and output material(s), in particular care composition ingredient(s), 104 of the chemical production network 102.

The production operating system 106 may be configured to register inbound environmental attributes and to assign outbound environmental attributes. The production operating system 106 may be configured to access data related the inputs materials 100, the processes and/or the output material(s), in particular care composition ingredient(s), 104 used in the chemical production network 102. For example, the production operating system 106 may be configured to register a recycled or bio-based content of the one or more input material(s) 100 used in the chemical production network 102 as environmental attribute. The production operating system 106 may be configured to allocate the environmental attribute to at least one balancing account associated with the recycled or bio-based content of the input materials 100. The production operating system 106 may be configured to allocate at least a part of the environmental attributes from the at least one balancing account to the at least one output material(s), in particular to the care composition ingredient(s), 104.

The production operating system 102 may be configured to handle environmental attributes related to the input materials 100 and output material(s), in particular care composition ingredient(s), 104 of the chemical production network 102. For example, the production operating system 106 may be configured to determine environmental attributes associated with the use of input materials 100 impacting the environmental property of the chemical production network 102 and the output material(s), in particular care composition ingredient(s) 104 produced by the chemical production network 102. Further in particular, the production operating system 102 may be configured to determine environmental attributes associated with the output material(s), in particular care composition ingredient(s) 104. This way the production operating system 102 may be configured to store environmental attributes in balancing accounts or to withdraw environmental attributes from the balancing accounts. The environmental attributes may hence be viewed as a credit that may be deposited in an account or deducted from an account related to the input and output material(s), in particular care composition ingredient(s), of the chemical production network 102. This way the environmental impact of the production may be tracked and/or traced.

In chemical production networks 102 multiple value chains may be linked. Additionally different input materials 100 or chemical processes impacting the environmental property of output material(s), in particular care composition ingredient(s), 104 produced by the chemical production network 102 may be used. Examples of input materials 100 impacting at least one environmental property of output material(s), in particular care composition ingredient(s) 104 produced from such input materials 100 are recycled, renewable or bio-based input materials 104. Examples of chemical processes impacting the environmental property include chemical processes using environmentally friendly technology such as carbon capture, carbon utilization or heat pumps.

Owing to the processing of chemicals in continuous or semi-continuous production and the complexity of chemical production networks 102, traceability of the input materials through the network may be hampered. In such scenarios, an equivalent environmental attribute signifying the impact on the environmental property of output material(s), in particular care composition ingredient(s) 104 produced by the chemical production network may be allocated to balancing accounts and assigned to one or more output material(s), in particular care composition ingredient(s), 104 of the chemical production network 102. The environmental attributes may hence be decoupled from the physical material flow inside the chemical production network 102. Decoupling may be based on the mass balance model in that the equivalent amount assigned to the one or more output material(s), in particular care composition ingredient(s) may not exceed the equivalent amount provided by input materials or processes. If an equivalent amount has been allocated to the virtual account of one environmental attribute type, it may not be allocated a second time to another virtual account of the one environmental attribute type. Environmental attribute types may be recycled, bio-based, renewable or the like. Environmental attributes may be provided in the form of digital assets or care composition ingredient passports attached to the physical entity of the care composition ingredient.

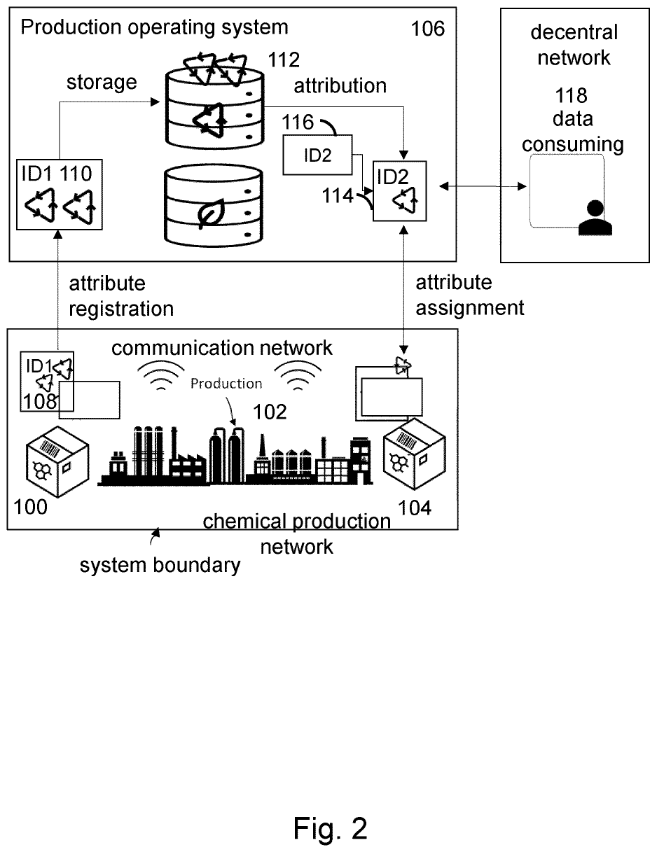

FIG. 2 illustrates schematically an example of attributing environmental attributes associated with input materials 100 to output material(s), in particular care composition ingredient(s) 104 of the chemical production network 102.

As shown in FIG. 1 the chemical production network 102 and operations of the chemical production network 102 may be monitored and/or controlled by a production operating system 106. The production operating system 106 may be configured to track environmental attributes from input materials 100 fed to the chemical production network 102 to output material(s), in particular care composition ingredient(s), 104 produced by the chemical production network 102. For tracking the operating system 106 may be configured to register environmental attributes associated with the input materials 100 provided to the chemical production network 102 and to attribute environmental attributes to output material(s), in particular care composition ingredient(s), 104 produced by the chemical production network 102.

The input materials 100 such as pyrolysis oil, bio-naphtha or bio-gas may be provided to the chemical production network 102. The input materials 100 may enter the chemical production network 104 at the entry point, such as a such as a steam cracker or a syngas plant. The input materials 100 may be used in the chemical production network 102 to produce one or more output material(s), in particular care composition ingredient(s), 104 from the input materials 100. output material(s), in particular care composition ingredient(s), 104 may be provided at exit points of the chemical production network 102. Further output material(s) may be MDI, TDI, PA6, EPS, PC, Polyols, Caprolactam, adipic acid, HMD, Polyamides.

On entry of the input material 100, input material data 108 may be provided via a communication network to a computing interface of the production operating system 106. A data provider, such as a QR code reader, may be configured to provide material data 108 related to the one or more input material(s) 100 and respective environmental attributes 108 to a computing interface configured to allocate the environmental attributes associated with the input materials 100. The material data 108 may include the input material identifier and environmental attributes associated with the input materials 100. The input material identifier may be associated with the physical entity of the input material 100 entering the chemical production network 102. The material data may be provided on, prior or after providing of the one or more input material(s) at entry points to the chemical production network 102.

The input material identifier may be linked to the environmental attribute(s) associated with the respective input material(s) 100, the amount of input material 100 and the certificate certifying the environmental attribute(s). The amount of input material may be a measured amount of input material 100 fed to a plant or storage of the chemical production network 102 for producing one or more output material(s), in particular care composition ingredient(s) 104 from the input material(s) 100. The input material identifier associated with the respective input material 100, the environmental attribute(s) associated with the respective input material(s) 100 and the amount of input material(s) 100 provided to the chemical production network 102 may be provided to the production operating system 106. Such data may be provided via a communication network on entry to chemical production network 102, or the data may be transferred from a computing system to the production operating system 106.

An inbound allocator 110 may be configured to allocate the one or more environmental attribute(s) to at least one balancing account 112 associated with the respective environmental attribute. For example, one balancing account 112 may relate to environmental attributes from recycled material and another balancing account 112 may relate to environmental attributes from bio-based material. The balancing account may be associated with the respective environmental attribute type, such as bio-based or recycled. Based on such association the balancing account associated with the environmental attribute type of the respective input material 100 may be selected. The environmental attributes may be allocated to the selected balancing account. For example, the account 112 for recycled material may be selected and the environmental attribute may be allocated to such account 122.

To allocate, the one or more environmental attribute(s) may be converted to balancing units and the balancing units may be allocated to the balancing account 122. The conversion may be based on a conversion factor such as mass, weight, carbon atoms, hydrogen atoms, methane equivalents or any other suitable measure for quantifying the environmental impact of the environmental attribute. The conversion factor may hence take into account the difference between producing chemical products from conventional input material(s) and producing chemical products from non-conventional input material(s) or producing chemical products from a mix of conventional and non-conventional input materials. The conversion factor may relate to differences in chemical and/or physical properties of conventional and non-conventional input material(s).

By using the balancing accounts 112 it can be ensured that environmental attributes of input materials 100 are only used once for assignment to care composition ingredients 104. This way double counting on input or output is avoided to ensure positive environmental impact can be reliable tracked and assigned to care composition ingredients 104.

An identifier provider 116 may be configured to provide the care composition ingredient identifier associated with the care composition ingredient produced by the chemical production network 102 and provided at the exit point from the chemical production network 102.

An outbound assigner 114 may be configured to assign at least one environmental attribute from the at least one balancing account 112 associated with the respective environmental attribute to the care composition ingredient identifier ID2. One or more environmental attribute(s) may be assigned to the at least one care composition ingredient identifier ID2. Assignment may include de-allocation of the one or more environmental attributes from the balancing account 112 associated with the respective environmental attribute type. Assignment may include converting one or more balancing unit(s) to one or more environmental attribute(s).

Assigning at least one environmental attribute associated with input material(s) to care composition ingredient(s) may include the linking of the care composition ingredients identifier ID2 with the environmental attribute. The care composition ingredient identifier ID2 may be associated with the physical entity of the care composition ingredient. This way the virtual identifier of a material may be uniquely linked to the physical material. Such linking may include a physical or virtual link of identifiers uniquely associated with the physical material. For physical linking a tag or code may be physically connected to the material, e.g., by printing a QR code on the packaging. For virtual linking different identifiers associated with the physical material may be linked. For example, an order number, a batch number, LOT number or a combination thereof may be linked.

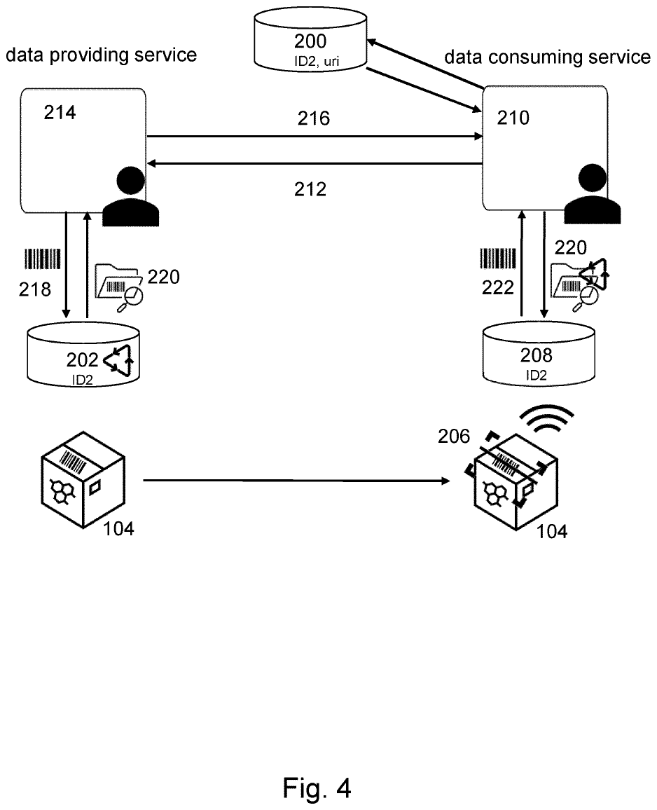

The outbound assigner 114 may be configured to provide the environmental attributes associated with the care composition ingredient to a data consumer, such as a system associated with a user of the care composition ingredient. The outbound assigner 114 may be configured to provide the environmental attributes associated with the care composition ingredient to a decentral network as will be described in the example of FIG. 4. Environmental attributes may be provided via the above ID based schema in the form of digital assets or care composition ingredient passports associated with the physical entity of the care composition ingredient.

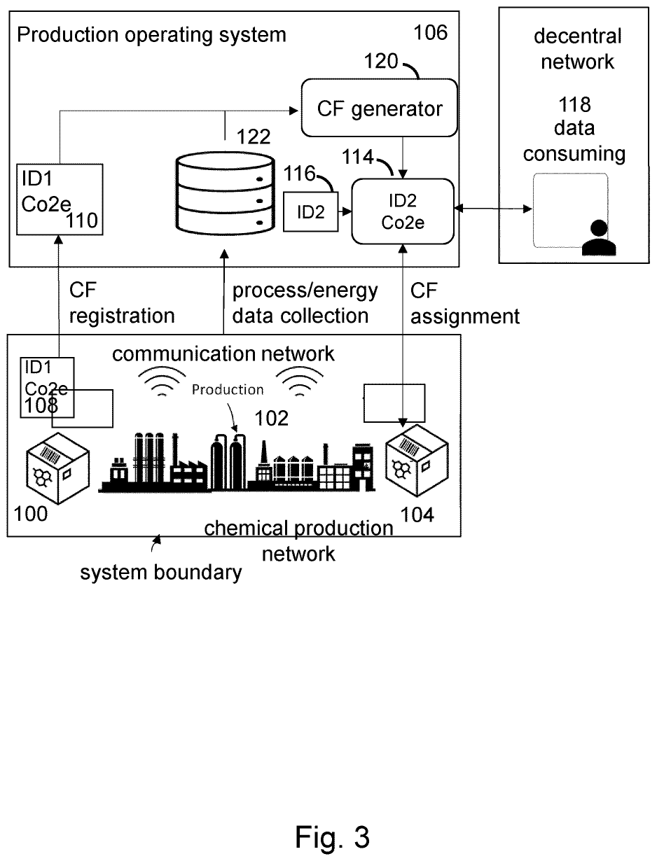

FIG. 3 illustrates schematically an example of attributing environmental attributes of input materials 100 and chemical processes to the care composition ingredient 104 of the chemical production network 102.

As described in the context of FIGS. 1 and 2 the chemical production network 102 and operations of the chemical production network 102 may be monitored and/or controlled by a production operating system 106. Input materials 100 such as pyrolysis oil, bio-naphtha or bio-gas may be provided to the chemical production network 102. The input materials 100 may be used in the chemical production network 102 to produce one or more care composition ingredient(s) 104 from the input materials 100.

On entry of the input material 100, input material data 108 may be provided via a communication network to a computing interface of the production operating system 106. A data provider, such as a QR code reader, may be configured to provide material data 108 related to the one or more input material(s) 100 and respective environmental attributes 108 to a computing interface configured to allocate the environmental attributes associated with the input materials 100. The material data 108 may include the input material identifier and environmental attributes associated with the input materials 100. The input material identifier may be associated with the physical entity of the input material 100 entering the chemical production network 102. The input material identifier may be linked to the carbon footprint of the input material 100 as environmental attribute. The material data may be provided on, prior or after providing of the one or more input material(s) at entry points to the chemical production network 102.

The inbound allocator 110 may be configured to retrieve the one or more environmental attribute(s) and to provide such attributes to the carbon footprint (CF) generator 120. A process data provider 122 may be configured to gather process data associated with the chemical processing of the input material(s) 100 to produce the care composition ingredient(s) 104. The process data provider 122 may be configured to gather energy data associated with the energy consumption of the chemical processing. The process data provider 122 may be configured to provide the process data and the energy data to the CF generator 120.

The CF generator 120 may be configured to determine the carbon footprint of the care composition ingredient produced by the chemical production network. The carbon footprint of the of the care composition ingredient may be determined based on the process data, the energy data and the carbon footprint of the input material(s) 100 used to produce the care composition ingredient.

An identifier provider 116 may be configured to provide the care composition ingredient identifier associated with the care composition ingredient produced by the chemical production network 102 and provided at the exit point from the chemical production network 102.

An outbound assigner 114 may be configured to assign the determined carbon footprint to the care composition ingredient identifier ID2. One or more environmental attribute(s) may be assigned to the at least one care composition ingredient identifier ID2, such as described in the context of FIG. 2.

The outbound assigner 114 may be configured to provide the environmental attributes, in particular the carbon footprint, associated with the care composition ingredient to a data consumer, such as a system associated with a user of the care composition ingredient. The outbound assigner 114 may be configured to provide the environmental attributes associated with the care composition ingredient to a decentral network as will be described in the example of FIG. 4. Environmental attributes may be provided via the above ID based schema in the form of digital assets or care composition ingredient passports associated with the physical entity of the care composition ingredient.

FIG. 4 illustrates schematically an example of a method or apparatus for providing environmental attributes associated with care composition ingredients to a material user as data consumer via a decentral network.

The care composition ingredient 104 as produced by the chemical production network 102 may be provided in association with the digital asset as described in the context of FIGS. 2 and 3. The digital asset may include the care composition ingredient identifier. The digital asset may include one or more environmental attribute(s) such as the product carbon footprint, recycled content or bio-based content. The digital asset may relate to one or more environmental attribute(s) such as the product carbon footprint, recycled content or bio-based content. The digital asset may include a digital representation of one or more environmental attribute(s) such as the product carbon footprint, recycled content or bio-based content.

The digital asset may further include or relate to authentication and/or authorization information linked to the care composition ingredient identifier. The authentication and/or authorization information may be provided for authentication and/or authorization of a data providing service 208 and/or data consuming service 210. The care composition ingredient identifier may include or relate to a decentral identifier, that is uniquely associated with the care composition ingredient. The decentral identifier may be connected to the digital representation of the environmental attributes. The digital representation may include a representation for accessing the environmental attributes or parts thereof. The decentral identifier may include a Universally Unique IDentifier (UUID) or a Digital IDentifier (DID). The decentral identifier may include any unique identifier uniquely associated with a data owner and/or care composition ingredient. The data owner may be the producer of the care composition ingredient. Via the decentral identifier and its unique association with the data owner and/or care composition ingredient access to the material configuration data may be controlled by the data owner.

The digital asset including the digital representation of one or more environmental attribute(s) such as the product carbon footprint, recycled content or bio-based content may be stored in a decentral data base 200. The one or more environmental attribute(s) such as the product carbon footprint, recycled content or bio-based content may be stored in a data base 202 associated with the data owner, such as the producer of the care composition ingredient 104.

The care composition ingredient 104 may be physically delivered to a user of the care composition ingredient. The care composition ingredient may be connected with a QR-code having encoded the care composition ingredient identifier. The user of the care composition ingredient may read the QR-code through a QR-code reader 206. The care composition ingredient identifier may be provided to a data base 208 associated with the consumer of the care composition ingredient 104. In other embodiments the consumer of the care composition ingredient may retrieve the care composition ingredient identifier through the decentral data base 200.

The data owner in this example may be the input material producer, the output material producer, the output material user, the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service 210 may comprise computer-executable instructions for accessing and/or processing data, such as care composition ingredient data, associated with the data owner. The data providing service 210 may comprise computer-executable instructions for providing and/or processing data, such as care composition ingredient data, associated with the data owner for accessing and/or processing by the data consuming service 214.

Based on the received care composition ingredient identifier a request to access the environmental attributes associated with the care composition ingredient identifier may be triggered by the data consuming service 210 as signified by arrow 212. The care composition ingredient identifier may be provided to the data providing service 214 associated with or of the producer of the care composition ingredient 104. In addition, authentication and/or authorization information may be provided.

The request may be authenticated and/or authorized to access the environmental attributes associated with the care composition ingredient identifier. Based on successful authorization and/or authentication access to the environmental attributes associated with the care composition ingredient identifier may be granted.

For access the care composition ingredient identifier may be provided to the data providing service 214 as signified by arrow 212. The data providing service 214 may use the received care composition ingredient identifier to retrieve the environmental attributes associated with the care composition ingredient 104 as signified by arrows 218 and 220. The environmental attributes associated with the care composition ingredient 104 provided to the data providing service 214 may be provided to the data consuming service 210 as signified by arrow 216. The environmental attributes associated with the care composition ingredient 104 may be stored in the data base 208 associated with the user of the care composition ingredient 104 as signified by arrow 220.

Through the output identifier or decentral identifier, the environmental attributes can be uniquely associated with the care composition ingredient. Through the decentral network the environmental attributes may be transferred between the producer of the care composition ingredient and the user of the care composition ingredient. This way the environmental attributes can be shared with unique association to the care composition ingredient and without central intermediary directly between the value chain players. This allows for transparency of environmental attributes across the value chain and positive environmental impacts from care composition ingredients produced by the chemical production network 102 can be tracked through the value chain.

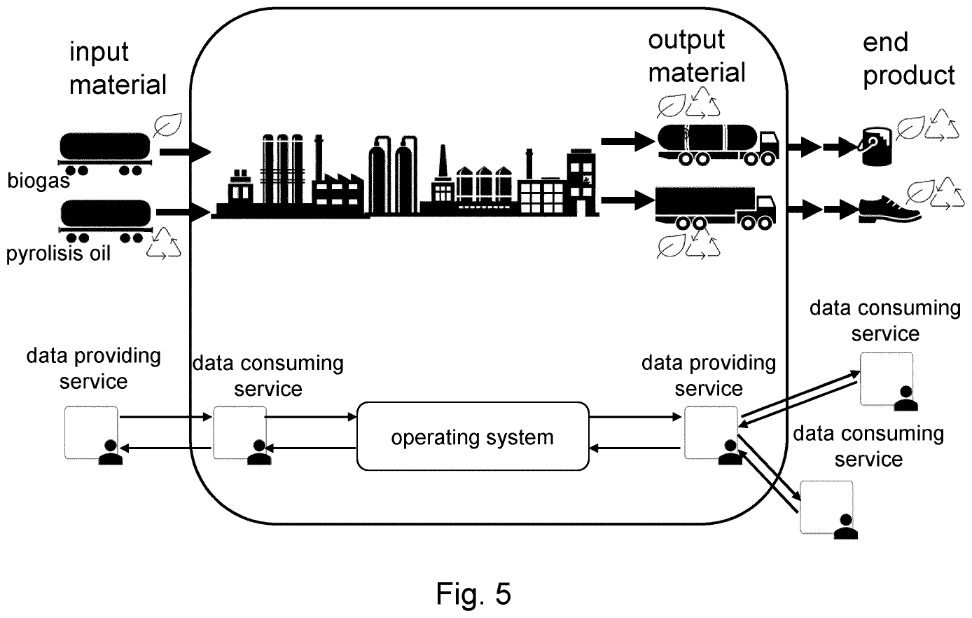

FIG. 5 illustrates schematically an example of a method or apparatus for providing environmental attributes associated with care composition ingredients across value chains via the decentral network.

In the example of FIG. 5 a fully connected value chain including the chemical production network is illustrated. In the example, the input material provider, the care composition ingredient producer, the care composition ingredient user and the end product producer may be connected to the decentral network as described in the context of FIG. 4. Environmental attributes may be provided via the ID based schema described in the context of FIGS. 2-4 in the form of digital assets or care composition ingredient passports associated with the physical entity of the input material, the care composition ingredient, any intermediate product or the end product.

The input material provider may provide the input materials such as bio-gas or pyrolysis oil. The environmental attributes of the input material may be provided through the data providing service connected to the decentral network as described in the context of FIG. 4. The care composition ingredient producer may produce the care composition ingredient from the input material(s) provided to the chemical production network. The care composition ingredient producer may access the environmental attributes associated with the input material through a data consuming service connected to the decentral network as described in the context of FIG. 4. The care composition ingredient producer may manage the environmental attributes via the production operating system as described in the context of FIGS. 1 to 3. The care composition ingredient producer may assign the environmental attributes associated with the input materials or environmental attributes associated with the chemical production network such as the carbon footprint, to the care composition ingredients as described in the context of FIGS. 1 to 3. The care composition ingredient producer may provide the environmental attributes associated with the care composition ingredient through the data providing service connected to the decentral network as described in the context of FIG. 4. The care composition ingredient user or the end product producer may access the environmental attributes associated with the care composition ingredient through the data consuming service connected to the decentral network as described in the context of FIG. 4.

The respective data owners in this example may be the input material producer, the output material producer, the output material user, the end product producer. The data owner may comprise any entity generating data. The data generating node may be coupled to the data owner or the entity owning or producing physical products from or for which data is generated. The data may be generated by a third-party entity on behalf of the entity owning physical products from or for which data is generated.

The data consuming service may comprise computer-executable instructions for accessing and/or processing data, such as care composition ingredient data, associated with the data owner. The data providing service may comprise computer-executable instructions for providing and/or processing data, such as care composition ingredient data, associated with the data owner for accessing and/or processing by the data consuming service.

In the example of FIG. 5 the decentral identifier may relate to the end product. Such decentral identifier may be provided to the value chain participants. Via the end product specific decentral identifier data associated with the end product produced from the care composition ingredient may be gathered across the production chain and assigned to the end product specific decentral identifier. For example, the one or more environmental attribute(s) associated with the end product may be derived from the environmental attribute(s) associated with the care composition ingredient, the input material or any other product entity present in the value chain of the end product.

This way the environmental attributes of input materials, care composition ingredients and any products produced from care composition ingredients may be tracked through the value chain up to the end product. By tracking the environmental attributes of materials in such way the information can be made transparent across the value chain while the information flow can be controlled by the participants in the supply chain. In addition, the environmental attributes can be handled according to the individual participants needs by production operating systems as described in the context of FIGS. 1 to 3. Overall, such tracking enables tracking of positive environmental impact by individual supply chain participants, which makes positive environmental impacts transparent and attributable to individual supply chain participants.

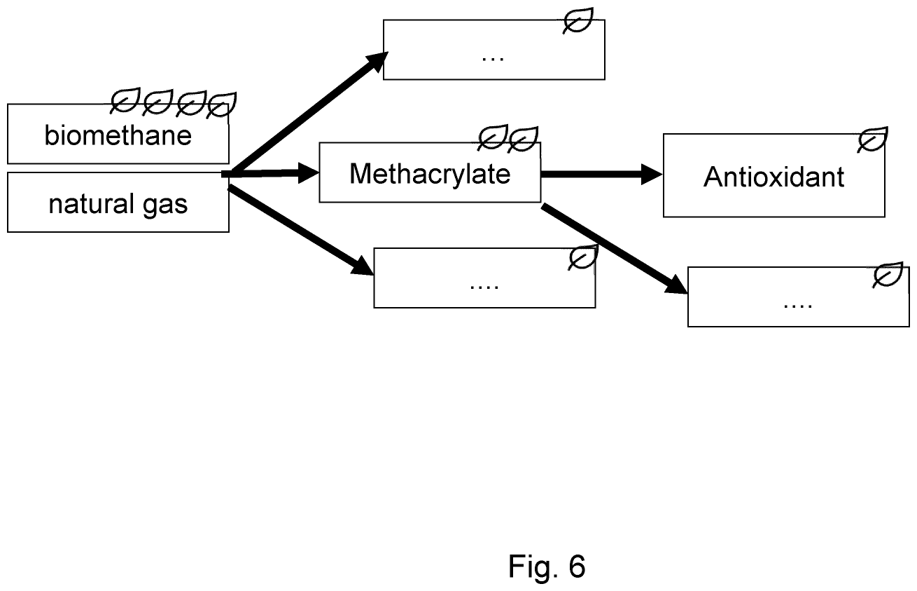

FIG. 6 illustrates schematically an example of a chemical production network for producing antioxidants associated with the digital asset. The example of FIG. 6 is an illustrative example and should not be considered limiting.

In the example of FIG. 6 natural gas and biomethane may be provided to the chemical production network for producing care composition ingredients such as anti-oxidants. Natural gas and biomethane may be the input materials in this example as mixed feedstock. From biomethane and natural gas methacrylate and other intermediate materials may be produced. From methacrylate, anti-oxidants and other products, such as intermediate materials or output materials, may be produced.

In the example of FIG. 6 the bio-based content of biomethane as non-fossil input material may be tracked. As described in the context of FIG. 2, the biomethane content at the start of the antioxidants production chain may be registered via the production operating system 106. Here the biomethane content may be allocated to the balancing account 112 for bio-based materials. The environmental attribute of biomethane content is hence detached from the mass flows of the chemical processing in the chemical production network 102 as illustrated in FIG. 6.

For attribution of the biomethane content used to produce the antioxidant, the biomethane content may be determined. The biomethane content attributable to anti-oxidant production may be based on mass conservation attributable to the produced anti-oxidant. For example, only half of the biomethane content may be attributable to the anti-oxidant and the other half may be attributable to other output products resulting from the biomethane as input material. The environmental attribute bio-based content may be attributed to such extend to the anti-oxidant.

In the system shown in FIG. 2, the environmental attribute associated with the production of antioxidant may be attributed to the produced anti-oxidant by associating the decentral ID to the anti-oxidant and assigning the environmental attribute to the decentral ID. By linking the ID and the environmental attribute, the environmental attribute may be uniquely linked to the produced anti-oxidant. The anti-oxidant may be delivered to the anti-oxidant user. The packaging such as lose bags with anti-oxidant may include a QR-code. The decentral ID may be included or encoded to the QR code. This way the anti-oxidant user may access the environmental attributes associated to the [anti-oxidant via the ID based protocol described in the context of FIGS. 4 and 5.

Similarly to this example the chemical production network for producing anti-oxidants associated with the digital asset may be based on the method illustrated in FIG. 3 for carbon footprints.

The present disclosure has been described in conjunction with preferred embodiments and examples as well. However, other variations can be understood and effected by those persons skilled in the art and practicing the claimed invention, from the studies of the drawings, this disclosure and the claims.

Any steps presented herein can be performed in any order. The methods disclosed herein are not limited to a specific order of these steps. It is also not required that the different steps are performed at a certain place or in a certain computing node of a distributed system, i.e. each of the steps may be performed at different computing nodes using different equipment/data processing.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action”includes any processing signal that triggers a computing node or device to perform the respective action.

In the claims as well as in the description the word “comprising” does not exclude other elements or steps and the indefinite article “a” or “an” does not exclude a plurality. A single element or other unit may fulfill the functions of several entities or items recited in the claims. The mere fact that certain measures are recited in the mutual different dependent claims does not indicate that a combination of these measures cannot be used in an advantageous implementation.

Any disclosure and embodiments described herein relate to the methods, the systems, devices, the computer program element lined out above and vice versa. Advantageously, the benefits provided by any of the embodiments and examples equally apply to all other embodiments and examples and vice versa.

All terms and definitions used herein are understood broadly and have their general meaning.

1. A system for producing a care composition ingredient associated with a digital asset, the system comprising:
a chemical production network configured to produce the care composition ingredient wherein the care composition ingredient is produced from one or more input material(s) through one or more chemical process(es) of the chemical production network, wherein the one or more input material(s) and/or the one or more chemical process(es) are associated with environmental attribute(s); and
a production operating apparatus configured to generate the digital asset by providing a decentral identifier associated with the produced care composition ingredient and linking the decentral identifier to the environmental attribute(s).

2. The system of claim 1, wherein the care composition ingredient associated with the digital asset is selected from the group consisting of acids, alkali carriers, antimicrobials, preservatives, biocides, anti-oxidants, anti-tarnish agents, bleach components, brighteners, pH buffers, builders, co-builders, catalytic agents, chelating agents, crystal growth inhibitors, clay soil removal/anti-redeposition agents, color speckles, color care agents, corrosion inhibitors, defoamers, desiccants, dispersants, dye transfer inhibiting agents, dyes and hueing agents, dye clay conjugates, emollients, enzymes, enzyme stabilizers, fabric softeners, humectants, hydrotopes, grease cleaning agents, light stabilizer, malodor control agents, opacifiers, perfumes, fragrances, perfume delivery systems, photocatalytic systems, pigments, polymers, soil release agents, preformed peracids, processing aids, rheology modifiers, scrubbing particles, silver care, soaps, solubilizing agents, solvents, peroxide sources, structure elasticizing agents, suds boosters, suds suppressors, surfactants, surface care agents, UV light stabilizers, waxes, and whitening agents.

3. The system of claim 1, wherein the digital asset of the care composition ingredient includes mass balanced environmental attributes related to the input material(s).

4. The system of claim 1, wherein the one or more environmental attribute(s) associated with the care composition ingredient are provided from at least one balancing account configured to store environmental attribute(s) associated with input material(s).

5. The system of claim 1, wherein the one or more environmental attribute(s) are associated with at least one property related to the environmental impact of the one or more input material(s) and/or the chemical process(es).

6. The system of claim 1, wherein the production operating apparatus is configured to gather environmental attributes associated with the produced care composition ingredient before, during and/or after production of the care composition ingredient by the chemical production network.

7. The system of claim 1, wherein the environmental attribute(s) associated with the care composition ingredient produced through chemical processes from one or more input material(s) provided to the chemical production network include the environmental at-tribute(s) associated with the input material(s), the chemical process(es) and/or the chemical production network(s).

8. The system of claim 1, wherein the environmental attribute(s) associated with input material(s) are provided before, during and/or after production of the care composition ingredient by the chemical production network, wherein the environmental attribute(s) associated with input material(s) are allocated to at least one balancing account before, during and/or after production of the care composition ingredient by the chemical production network.

9. The system of claim 1, wherein the environmental attribute(s) associated with the produced care composition ingredient relate to environmental properties generated from process data associated with the chemical processing of the input material(s) and/or energy data associated with the energy consumption of the chemical processing, wherein the environmental attribute(s) associated with the produced care composition ingredient are generated before, during and/or after production of the care composition ingredient by the chemical production network.

10. A method for producing a care composition ingredient associated with a digital asset, wherein the method comprises:
producing the care composition ingredient from one or more input material(s) through one or more chemical process(es) of a chemical production network, wherein the one or more input material(s) and/or the one or more chemical process(es) are associated with environmental attribute(s);
generating the digital asset by
providing a decentral identifier associated with the produced care composition ingredient and one or more environmental attribute(s) associated with the one or more in-put material(s) and/or the one or more chemical process(es);
linking the decentral identifier and the one or more environmental attribute(s); and
providing the produced care composition ingredient in association with the digital asset.

11. A care composition ingredient associated with a digital asset including a decentral identifier associated with the care composition ingredient and linked to one or more environmental attribute(s) of the one or more input material(s) and/or the one or more chemical process(es) used to produce the care composition ingredient.

12. The care composition ingredient of claim 11, wherein the care composition ingredient is selected from the group consisting of acids, alkali carriers, antimicrobials, preservatives, biocides, anti-oxidants, anti-tarnish agents, bleach components, brighteners, pH buffers, builders, co-builders, catalytic agents, chelating agents, crystal growth inhibitors, clay soil removal/anti-redeposition agents, color speckles, color care agents, corrosion inhibitors, defoamers, desiccants, dispersants, dye transfer inhibiting agents, dyes and hueing agents, dye clay conjugates, emollients, enzymes, enzyme stabilizers, fabric softeners, humectants, hydro-topes, grease cleaning agents, light stabilizer, malodor control agents, opacifiers, perfumes, fragrances, perfume delivery systems, photocatalytic systems, pigments, polymers, soil release agents, preformed peracids, processing aids, rheology modifiers, scrubbing particles, silver care, soaps, solubilizing agents, solvents, peroxide sources, structure elasticizing agents, suds boosters, suds suppressors, surfactants, surface care agents, UV light stabilizers, waxes, and whitening agents.

13. A method for generating a digital asset associated with a care composition ingredient, wherein the care composition ingredient is produced from one or more input material(s) through one or more chemical process(es) of a chemical production network, wherein the one or more input material(s) and/or the one or more chemical process(es) are associated with environmental attribute(s), the method comprising:
providing a decentral identifier associated with the produced care composition ingredient and one or more environmental attribute(s) of the one or more input material(s) and/or the one or more chemical process(es) used to produce the care composition ingredient;
linking the decentral identifier and the environmental attribute(s); and
providing the digital asset in association with the produced care composition ingredient, wherein the environmental attribute(s) associated with the care composition ingredient is made accessible to a user of the care composition ingredient through the digital asset.

14. A digital asset generated according to the method of claim 13.

15. A method for using the digital asset generated according to the method of claim 13, the method comprising using the digital asset in production of a product produced from the care composition ingredient associated with the digital asset.

16. A method for using the care composition ingredient of claim 11 associated with the digital asset, the method comprising using the care composition ingredient for producing a product from the care composition ingredient and deriving a digital asset associated with the product from the care composition ingredient digital asset.
